(19) United States
(12) Patent Application Publication
(10) Pub. No.: US 2011/0172168 A1
(43) Pub. Date: Jul. 14, 2011

(54) ILT-2 (LIR1) VARIANTS WITH INCREASED AFFINITY FOR MHC CLASS I MOLECULES

(75) Inventors: Bent Karsten Jakobsen, Oxfordshire (GB); Yi Li, Oxfordshire (GB); Ruth Karen Moysey, Oxfordshire (GB)

(73) Assignee: MediGene Ltd., Abingdon, Oxfordshire (GB)

(21) Appl. No.: 12/516,141

(22) PCT Filed: Nov. 24, 2006
(86) PCT No.: PCT/GB2006/004411

§ 371 Date: Sep. 30, 2009

(51) Int. Cl. A61K 38/17; C07K 14/54; C07H 21/04; C12N 15/63; C07K 14/435; A61P 37/06

(52) U.S. Cl. 514/21.2; 530/351; 536/23.5; 435/320.1; 530/350; 530/410; 530/402

(57) ABSTRACT

The present invention provides ILT-2 variants having the property of binding to a given Class IpMHC with a K_{D} of less than or equal to 1 μM and/or an off-rate (K_{off}) of 2 S⁻¹ or slower AND said polypeptide has at least a 45% identity and/or 55% similarity to SEQ ID NO: 19 AND said polypeptide inhibits CD8 binding to the given pMHC to a greater extent than the polypeptide SEQ ID NO: 3, CHARACTERISED IN THAT said polypeptide comprises an amino acid sequence selected from one of SEQ ID NOs 6 to 69. Such polypeptides are useful, either alone or associated with a therapeutic agent, for the inhibition of cyto-toxic T cell (CTL) responses.

The present invention relates to polypeptides having the property of binding to a given Class I pMHC with a K_{D} of less than or equal to 1 μM and/or an off-rate (k_{off}) of 2 S⁻¹ or slower AND said polypeptide has at least a 45% identity and/or 55% similarity to SEQ ID NO: 19 AND said polypeptide inhibits CD8 binding to the given pMHC to a greater extent than the polypeptide SEQ ID NO: 3, CHARACTERISED IN THAT said polypeptide comprises an amino acid sequence selected from one of SEQ ID NOs 6 to 69. Also provided are multivalent complexes of said polypeptides, cells presenting said polypeptides, said polypeptides associated with therapeutic agents and methods for using these polypeptides.

### BACKGROUND TO THE INVENTION

Immunoglobulin-like transcripts (ILTs) are also known as Leukocyte Immunoglobulin-like receptors (LIRs), monocyte/macrophage immunoglobulin-like receptors (MIRs) and CD85. This family of immunoreceptors form part of the immunoglobulin superfamily. The identification of ILT molecules was first published in March 1997 in a study (Samaridis et al., (1997) Eur J Immmmol 27 660-665) which detailed the sequence of LIR-1 (ILT-2), noted their similarity to bovine FCγ2R, human killer cell inhibitory receptors (KIRs), human FcαR, and mouse gp49. This study also noted that LIR-1, unlike KIRs, is predominately expressed on monocytic and B lymphoid cells.

Soluble polypeptides with the pMHC binding characteristics of ILT molecules and multivalent complexes thereof provide a means of blocking the CD8 binding site on pMHC molecules, for example for the purpose of inhibiting CD8⁺ T cell-mediated automimmune disease. However, for that purpose it would be desirable if these polypeptides had a higher affinity and/or a slower off-rate for the target pMHC molecules than native ILT molecules.

Our co-pending application PCT/GB2006/001860 describes and claims polypeptides having the property of binding to a given Class I pMHC CHARACTERISED IN THAT said polypeptide has a K_{D} for the said given Class I pMHC of less than or equal to 1 μM and/or has an off-rate (k_{off}) for the said given Class I pMHC molecule of 2 S⁻¹ or slower AND said polypeptide has at least a 45% identity and/or 55% similarity to SEQ ID NO: 7 AND said polypeptide inhibits CD8 binding to the given pMHC to a greater extent than the polypeptide SEQ ID NO: 3.

### BRIEF DESCRIPTION OF THE INVENTION

The present invention relates to polypeptides having the property of binding to a given Class I pMHC with a K_{D} of less than or equal to 1 μM and/or an off-rate (k_{off}) of 2 S⁻¹ or slower AND said polypeptide has at least a 45% identity and/or 55% similarity to SEQ ID NO: 19 AND said polypeptide inhibits CD8 binding to the given pMHC to a greater extent than the polypeptide SEQ ID NO: 3, CHARACTERISED IN THAT said polypeptide comprises an amino acid sequence selected from one of SEQ ID NOs 6 to 69. Also provided are multivalent complexes of said polypeptides, cells presenting said polypeptides, said polypeptides associated with therapeutic agents and methods for using these polypeptides.

### DETAILED DESCRIPTION OF THE INVENTION

As noted above ILT molecules are also known as LIRs, MIRs and CD85. The term ILT as used herein is understood to encompass any polypeptide within this family of immunoreceptors.

### ILTs

The ILT family of immunoreceptors are expressed on the surface of lymphoid and myeloid cells. The ILT molecules share 63-84% homology in their extracellular regions and all except the soluble LIR-4 are type I transmembrane proteins. All the currently identified ILT molecules have either two or four immunoglobulin superfamily domains in their extracellular regions. (Willcox et al., (2003) 4 (9) 913-919) Individual ILT molecules may also be expressed as a number of distinct variants/isoforms. (Colonna et al., (1997) J Exp Med 186 (11) 1809-1818) and (Cosman et al., (1997) Immunity 7 273-282)

There are a number of scientific papers detailing the structure and function of ILT molecules including the following: (Samaridis et al., (1997) Eur J Immunol 27 660-665), (Cella, et al., (1997) J Exp Med 185 (10) 1743-1751), (Cosman et al., (1997) Immunity 7 273-282), (Borges et al., (1997) J Immunol 159 5192-5196), (Colonna et al., (1997) J Exp Med 186 (11) 1809-1818), (Colonna et al., (1998) J Immunol 160 3096-3100), (Cosman et al., (1999) Immunological Revs 168 177-185), (Chapman et al., (1999) Immunity 11 603-613), (Chapman et al., (2000) Immunity 12 727-736), (Willcox et al., (2002) BMC Structural Biology 2 6), (Shiroshi et al., (2003) PNAS 100 (5) 8856-8861) and (Willcox et al., (2003) 4 (9) 913-919).

WO9848017 discloses the genetic sequences encoding ILT family members and their deduced amino acid sequences. This application classified LIR molecules into three groups. The first group containing polypeptides with a transmembrane region including a positively charged residue and a short cytoplasmic tail. The second group comprising polypeptides having a non-polar transmembrane region and a long cytoplasmic tail. And finally a third group containing a polypeptide expressed as a soluble polypeptide having no transmembrane region or cytoplasmic tail. Also disclosed were processes for producing polypeptides of the LIR family, and antagonistic antibodies to LIR family members. This application discussed the possible use of LIR family members to treat autoimmune diseases and disease states associated with suppressed immune function. In this regard, it was noted that the use of soluble forms of an LIR family member is advantageous for certain applications. These advantages included the ease of purifying soluble forms of ILTs/LIRs from recombinant host cells, that they are suitable for intravenous administration and their potential use to block the interaction of cell surface LIR family members with their ligands in order to mediate a desirable immune function. The possible utility of soluble LIR fragments that retain a desired biological activity, such as binding to ligands including MHC class I molecules was also noted.

Another study (Shiroishi et al (2003) PNAS 100 (15) 8856-8861) discussed soluble (truncated) forms of ILT-2 and ILT-4 molecules. Their ability to compete with soluble CD8 for binding to MHC molecules in Biacore studies was noted and it was postulated that this may be one of the mechanisms by which ILT-2 modulates CD8+ T cell activation. In relation to pMHC binding this study states “The higher affinity of ILT versus CD8 binding suggests that ILTs may effectively block CD8 binding at the cell surface. This study noted that ILT2 binds to the α3 domain of Class I MHC and that the crystal structure of an ILT2 fragment containing domains 1 and 2 had been reported.

(Colonna et al., (1998) J. Immunol. 160 3096-3100) which focussed on ILT-4, contains a summary of the tissue distribution and specificity of ILTs 2-5. Of these ILT molecules, ILT-2 and ILT-4 are noted to bind Class I MHC molecules. This study analysed the binding of soluble ILT-4 to cells transfected with various Class I MHCs. The study concluded that ILT-4 binds to HLAs-A, B and G, but not HLA-Cw3 or HLA-Cw5.

WO03041650 discloses a method of treating Rheumatoid Arthritis (RA) using modulators of LIR-2 and/or LIR-3/LIR-7 activity. The modulators disclosed include both agonists and antagonists of LIR activity. WO2006033811 discloses the use of ILT-3 polypeptides and fusions thereof as therapeutic agents for the inhibition of graft rejection.

The affinity for various soluble analogues of Wild-Type ILT molecules for different pMHC targets has been determined. For example, (Chapman et al., (1999) Immunity 11 603-613) used Biacore-based methods to determine that LIR-1 (ILT-2) bound to a range of HLA-A, HLA-B, HLA-C, HLA-E and HLA-G molecules. The determined K_{D} values for these interactions ranged from 1×10⁻⁴ M (for HLA-G1) to 2×10⁻⁵ M (for HLA-Cw*0702). This study also noted that the K_{D} of the interaction between ILT-2 had an affinity for UL18, a viral analogue of Class I MHC, in the nM range.

A further study (Chapman et al., (2000) Immunity 12 727-736) reported the crystal structure of a truncated LIR-1 (ILT-2) polypeptide comprising the D1 and D2 domains. LIR-1 was known to bind to the UL18 viral class I MHC analogue with much higher affinity than the similar LIR-2. The authors used the crystal structure of the truncated LIR-1 polypeptide to identify differences between LIR-1 and LIR-2 that occurred in solvent-exposed residues. Site-directed mutagenesis of these two peptides was the used to confirm which residues were involved in UL18 binding. This was carried out by substituting WT residues from LIR-1 in to the corresponding amino acid positions of LIR-2. The study concluded that residue 38Y, and at least one of 76Y, 80D or 83R of LIR-1 were involved in UL18 binding. The authors stated that “Because the affinity of LIR-1 for class I MHC proteins is much lower than for UL18 we were unable to derive accurate affinities for the binding of the LIR-1 and LIR-2 mutants to class I MHC.”

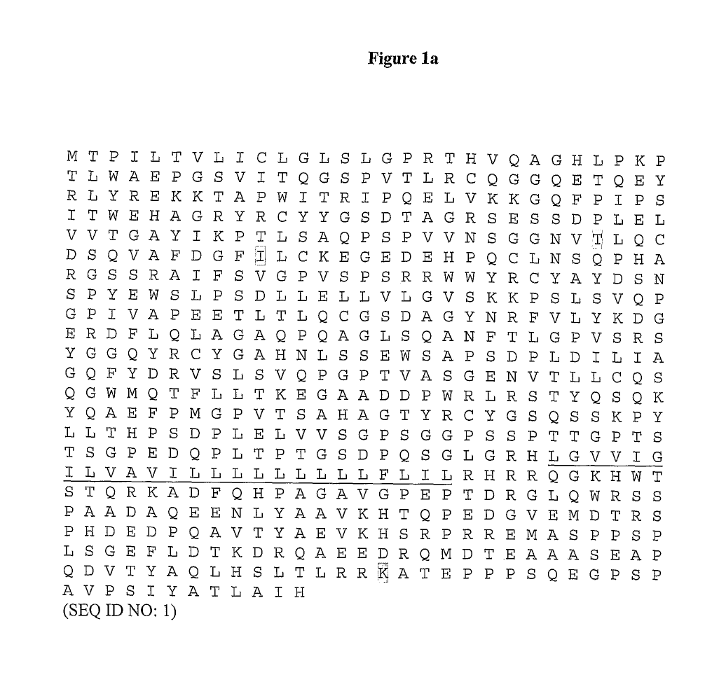

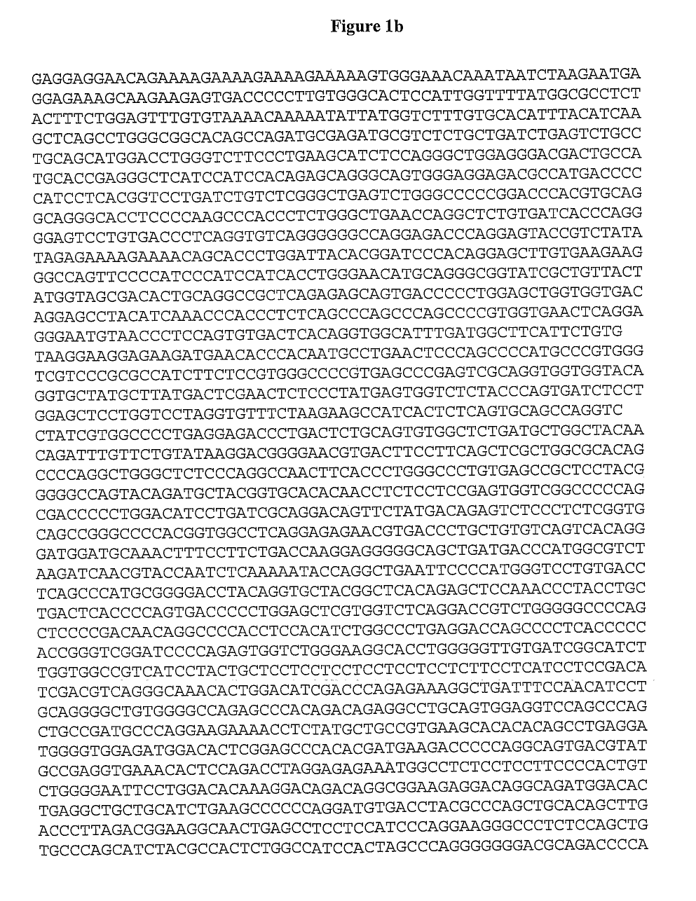

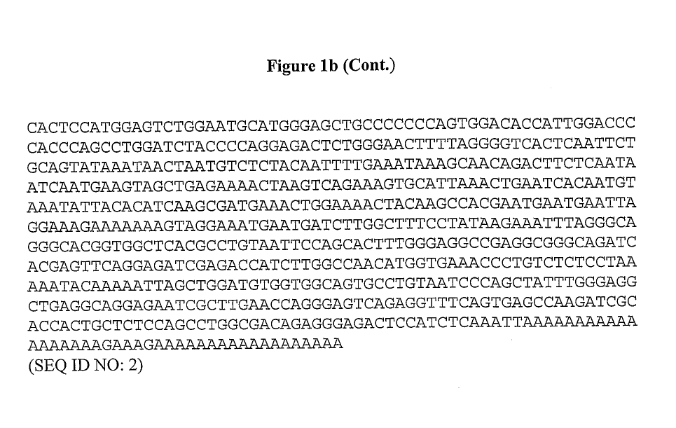

The full amino acid and DNA sequences of a Wild-Type human ILT-2 are shown in FIGS. 1a (SEQ ID NO:1) and 1b (SEQ ID NO:2) respectively. The DNA sequence provided corresponds to that given accession number NM_{—}006669 on the NCBI nucleotide database.

### High Affinity ILT-Like Polypeptides

This invention provides additional high affinity ILT-like polypeptides within the scope of, but not disclosed in, our co-pending application PCT/GB2006/001860.

The present invention provides polypeptides having the property of binding to a given Class I with a K_{D} of less than or equal to 1 μM and/or an off-rate (k_{off}) of 2 S⁻¹ or slower AND said polypeptide has at least a 45% identity and/or 55% similarity to SEQ ID NO: 19 AND said polypeptide inhibits CD8 binding to the given pMHC to a greater extent than the polypeptide SEQ ID NO: 3, CHARACTERISED IN THAT said polypeptide comprises an amino acid sequence selected from one of SEQ ID NOs 6 to 69. Certain embodiments of the present aspect are provided by variations of these polypeptides which have 196L and 198L instead of 196D and/or 198D, using the numbering of SEQ ID NO: 3.

Polypeptides which meet the above homology and Class I pMHC-binding criteria may be regarded as high affinity ILT-like molecules and may be referred to herein as such.

As stated above, naturally occurring ILT polypeptides have either two or four immunoglobulin superfamily domains in their extracellular regions. The high affinity ILT-like polypeptides of the invention may be expressed in forms having four, three or two of said domains. The currently preferred embodiments of the invention have two immunoglobulin superfamily domains corresponding to the two N-terminal domains of human ILT-2 containing one or more mutation(s) which confer high affinity for Class I pMHC. These N-terminal domains are domains one and two using the notation of Cosman et al., (1999) Immunol Revs 168: 177-185. ILT-like polypeptides having those two N-terminal domains generally have a sequence corresponding to amino acids 1-195 of SEQ ID NO: 3.

One embodiment of the invention is provided wherein the polypeptide is a mutated human ILT molecule. For example, the DNA encoding human ILT-2, or soluble fragments thereof, can be used as a template into which the various mutations that cause high affinity and/or a slow off-rate for the interaction between the high affinity ILT-like polypeptides of the invention and the target pMHC complex can be introduced. Thus the invention includes ILT-2 variants which are mutated relative to the native sequence.

As will be obvious to those skilled in the art the mutation(s) in such human ILT-2 amino acid sequence may be one or more of substitution(s), deletion(s) or insertion(s).

These mutations can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) mutagenesis and restriction enzyme-based cloning see (Sambrook & Russell, (2001) Molecular Cloning—A Laboratory Manual (3′ Ed.) CSHL Press) Further information on LIC procedures can be found in (Rashtchian, (1995) Curr Opin Biotechnol 6 (1): 30-6)

Another embodiment is provided by a polypeptide of the invention comprising amino acids corresponding to at least amino acids 1-195 of SEQ ID No: 3. Such polypeptides are two-domain embodiments comprising domains corresponding to the two N-terminal immunoglobulin superfamily domains of human ILT-2.

Another aspect of the invention is provided by mutated human ILT-2 polypeptides having the property of binding to a given Class I pMHC with a K_{D} of less than or equal to 1 μM and/or an off-rate (k_{off}) of 2 S⁻¹ or slower AND said polypeptide has at least a 45% identity and/or 55% similarity to SEQ ID NO: 19 AND said polypeptide inhibits CD8 binding to the given pMHC to a greater extent than the polypeptide SEQ ID NO: 3, CHARACTERISED IN THAT one or more of amino acids corresponding to amino acids 23V, 35E, 126Q, 127V, 128A, 129F, 130D, 170R, 179D, 180S, 181N, 182S, 187S or 189P of SEQ ID NO: 3 is/are mutated. Certain embodiments of the present aspect include mutated human ILT-2 polypeptides comprising one or more of the following mutations 23V→L, 35E→Q, 126Q→P, 126Q→M, 127V→W, 127V→F, 128A→D, 128A→S, 128A→T, 128A→Y, 128A→N, 128A→L, 128A→Q, 128A→I, 129F→A, 129F→T, 129F→S, 129F→V, 130D→E, 170R→K, 179D→P, 179D→N, 179D→M, 179D→T, 179D→G, 180S→I, 180S→A, 180S→N, 180S→D, 180S→W, 180S→R, 180S→E, 181N→W, 181N→F, 181N→Y, 182S→T, 182S→A, 182S→W, 182S→F, 182S→L, 187S→T and 189P→G, 189P→M or 189P→S using the numbering of SEQ ID NO: 3. For example, polypeptides comprising at least two, three, four, five, six, seven, eight or nine of the above mutations will often be suitable.

One preferred embodiment of the present aspect is provided by a mutated human ILT-2 polypeptide comprising mutations corresponding to 19Q→M, 20G→D, 21→Q, 83A→S, 85R→W, 87E→A, 99A→N, 179D→M, 181N→W, 182S→A, 196L→D and 198L→D using the numbering of SEQ ID NO: 3

Another aspect of the invention is provided by a mutated human ILT-2 polypeptide having the property of binding to a given Class I pMHC with a K_{D} of less than or equal to 1 μM and/or an off-rate (k_{off}) of 2 S⁻¹ or slower AND said polypeptide has at least a 45% identity and/or 55% similarity to SEQ ID NO: 19 AND said polypeptide inhibits CD8 binding to the given pMHC to a greater extent than the polypeptide SEQ ID NO: 3, comprising one or more of mutations corresponding to 188L→R, 188L→S 188L→T and 188L→Q using the numbering of SEQ ID NO: 3.

Preferably, the polypeptide is CHARACTERISED IN THAT said polypeptide has at least a 60% identity and/or 75% similarity to SEQ ID NO: 19

Preferably, the polypeptide is CHARACTERISED IN THAT said polypeptide has at least a 75% identity and/or 85% similarity to SEQ ID NO: 19.

Preferably, the polypeptide is CHARACTERISED IN THAT said polypeptide has at least a 90% identity and/or 95% similarity to SEQ ID NO: 19.

Sequence identity as used herein means identical amino acids at corresponding positions in the two sequences which are being compared. Similarity in this context includes amino acids which are identical and those which are similar (functionally equivalent). For example a single substitution of one hydrophobic amino acid present at a given position in a polypeptide with a different hydrophobic amino acid would result in the formation of a polypeptide which was considered similar to the original polypeptide but not identical). The parameters “similarity” and “identity” as used herein to characterise polypeptides of the invention are determined by use of the FASTA algorithm as implemented in the FASTA programme suite available from William R. Pearson, Department of Biological Chemistry, Box 440, Jordan Hall, Charlottesville, Va. The settings used for determination of those parameters via the FASTA programme suite are as specified in Example 6 herein.

As will be obvious to those skilled in the art there are a number of sources of FASTA protein: protein comparisons which could be used for this analysis. (Pearson et al., (1988) PNAS 85 2444-2448) provides further details of the FASTA algorithm. The relative inhibitory activities of the polypeptide of SEQ ID NO 3 and any given putative polypeptide of the invention may be determined by any conventional assay from which the read-out is related to the binding affinity of CD8 for the given pMHC. In general the read-out will be an IC₅₀ value. The test polypeptide and that of SEQ ID NO: 3 will be assessed at comparable concentrations and their respective IC₅₀'s determined by reference to the inhibition curves plotted from the individual results. A suitable assay is that described in Example 5.

Preferably, the polypeptide is CHARACTERISED IN THAT said polypeptide has a K_{D} for the said given Class I pMHC of less than or equal to 100 nM and/or has an off-rate (k_{off}) of 0.1 S⁻¹.

As will be known to those skilled in the art there are a number of means by which said affinity and/or off-rate can be determined. For example, said affinity (K_{D}) and/or off-rate (k_{off}) may be determined by Surface Plasmon Resonance. Example 4 herein provides a Biacore-based assay suitable for carrying out such determinations

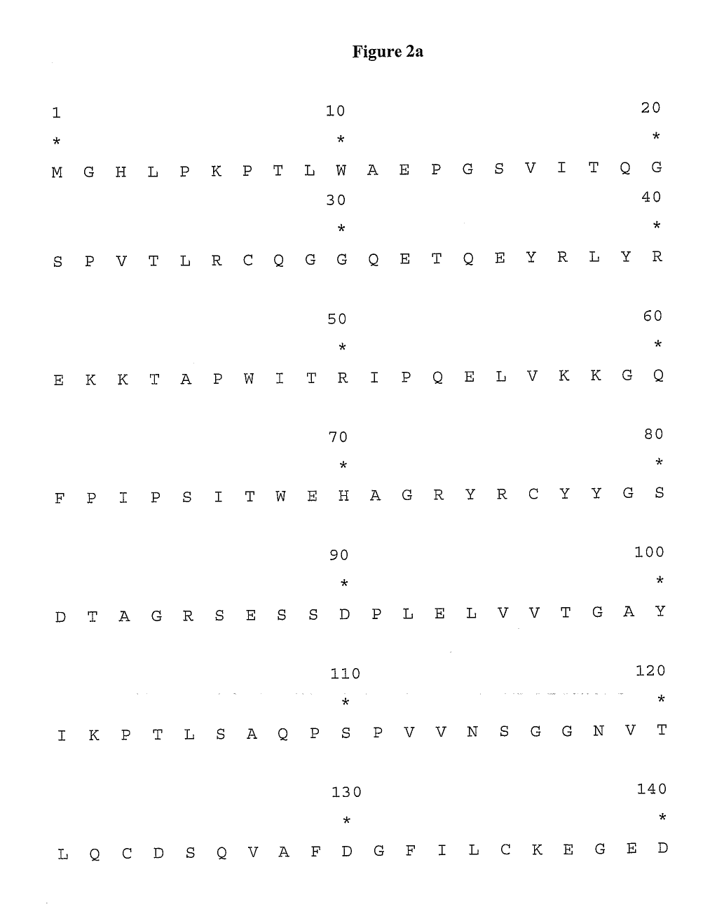

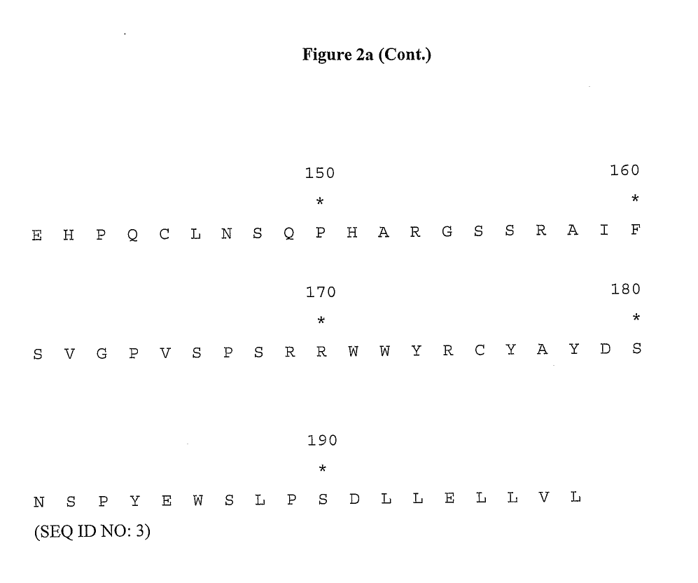

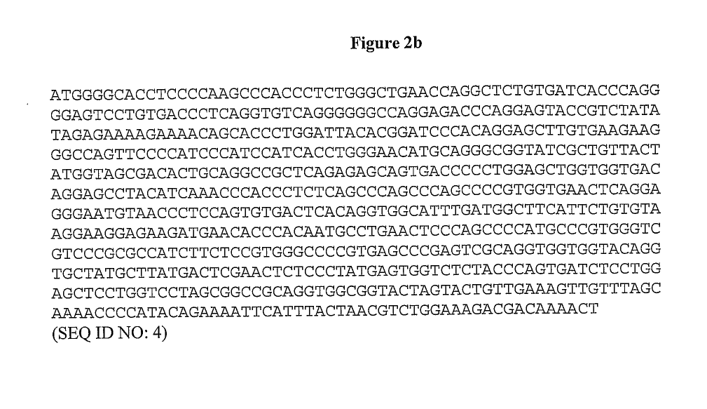

For comparison the interaction of a soluble truncated variant of the Wild-Type ILT-2 molecule (see FIG. 2a (SEQ ID NO: 3) for the amino acid sequence of this soluble polypeptide) and HLA-A*0201 loaded with the Carcinoembryonic antigen (CEA)-derived YLSGANLNL (SEQ ID NO: 71) peptide has a K_{D} of 6 μM, and an off-rate (k_{off}) of 2.4 S⁻¹ as measured by the Biacore-based method of Example 4. This soluble ILT-2 molecule is a truncated form of a variant of isoform 1 of Wild Type human ILT-2 which contains only extracellular domains D1 and D2. The amino acid residues which differ between this ILT-2 variant molecule and those of isoform 1 of ILT-2 are highlighted in FIG. 1a.

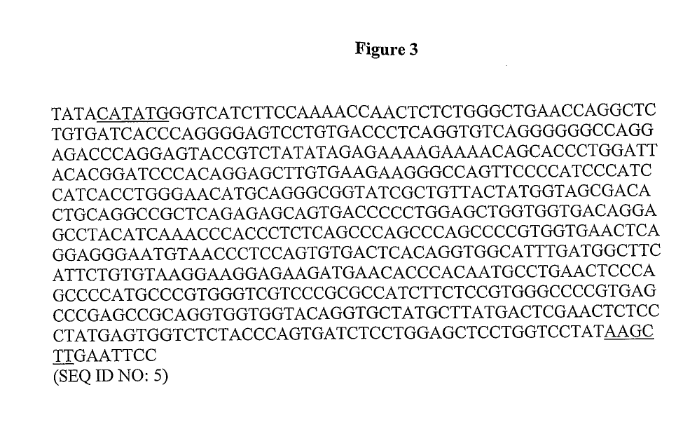

FIG. 2b (SEQ ID NO: 4) details the native DNA sequence encoding this polypeptide. In order to improve the efficiency of recombinant expression and to facilitate cloning of this polypeptide a number of mutations were introduced into the DNA encoding this polypeptide. These mutations do not alter the amino acid sequence of the expressed polypeptide. The DNA sequence used for recombinant expression is shown in FIG. 3 (SEQ ID NO: 5)

One embodiment of the invention is provided wherein the polypeptide is a mutated human ILT molecule. For example, the DNA encoding human ILT-2, or soluble fragments thereof, can be used as a template into which the various mutations that cause high affinity and/or a slow off-rate for the interaction between the high affinity ILT-like polypeptides of the invention and the target pMHC complex can be introduced. Thus the invention includes ILT-2 variants which are mutated relative to the native sequence.

As will be obvious to those skilled in the art the mutation(s) in such human ILT-2 amino acid sequence may be one or more of substitution(s), deletion(s) or insertion(s). These mutations can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) mutagenesis and restriction enzyme-based cloning see (Sambrook & Russell, (2001) Molecular Cloning—A Laboratory Manual (3^{rd} Ed.) CSHL Press) Further information on LIC procedures can be found in (Rashtchian, (1995) Curr Opin Biotechnol 6 (1): 30-6)

For example, polypeptides comprising at least two, three, four, five, six, seven, eight, or nine of the above mutations will often be suitable.

The numbering used is the same as that shown in FIG. 2a (SEQ ID No: 3).

Another embodiment is provided by a polypeptide of the invention comprising amino acids corresponding to at least amino acids 1-195 of SEQ ID No: 3. Such polypeptides are two-domain embodiments comprising domains corresponding to the two N-terminal immunoglobulin superfamily domains of human ILT-2.

Those skilled in the art will appreciate that it is inevitable that there will be minor amino acid substitutions, deletions and insertions which do not affect the overall identity and properties of the embodiment. In particular, it should be noted that truncations of 1, 2, 3, 4 or 5 amino acids at the N-terminus of the polypeptides of the inventions are unlikely to impair the functionality of the polypeptide. Such minor variations may be regarded as phenotypically silent variations of such polypeptides. Looked at another way, such variations result in a polypeptide which has the same function as the parent and achieves that function in the same way,

A preferred embodiment of the invention is provided by a polypeptide which consists of or includes SEQ ID No: 19.

High Affinity ILT-Like Polypeptides with Enhanced Solubility

The polypeptides of the invention may be used as soluble therapeutics. In such instances is desirable to increase the solubility of these polypeptides. The invention encompasses polypeptides which comprise one or more mutation(s) which increase the solubility of the polypeptide relative to a corresponding polypeptide lacking said mutations. As will be known to those skilled in the art when increased solubility of a polypeptide is sought it is generally preferable to mutate amino acids which are solvent exposed. These solvent exposed amino acids can be identified by reference to the crystal structure of ILT-2. (See Chapman et al., (2000) Immunity 12 727-736) The invention encompasses polypeptides wherein one or more solvent-exposed amino acid(s) are mutated. For example, polypeptides of the invention comprising at least one mutation wherein a solvent exposed hydrophobic amino acid is substituted by a charged amino acid.

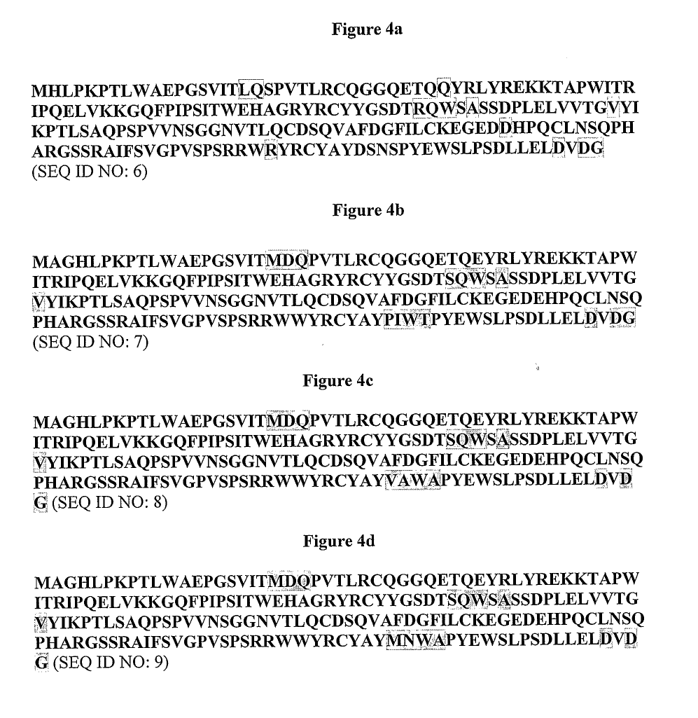

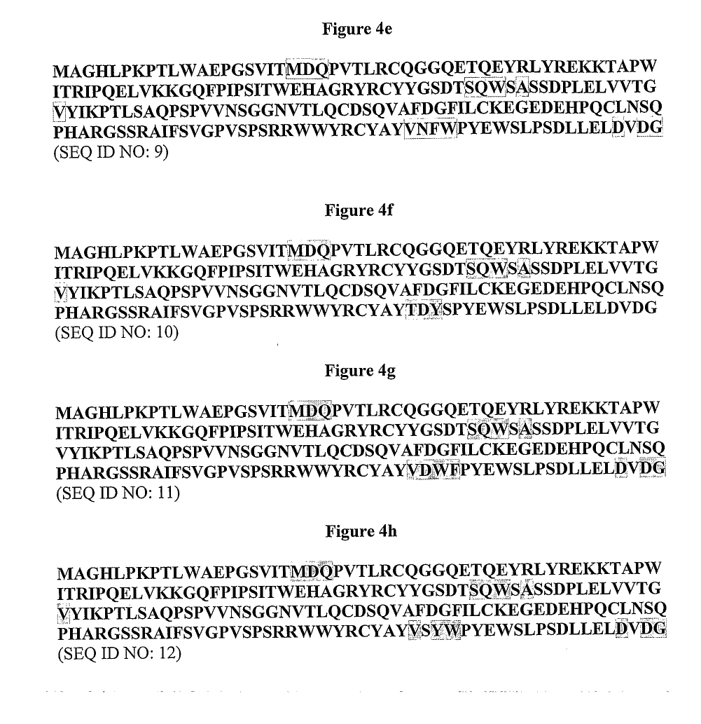

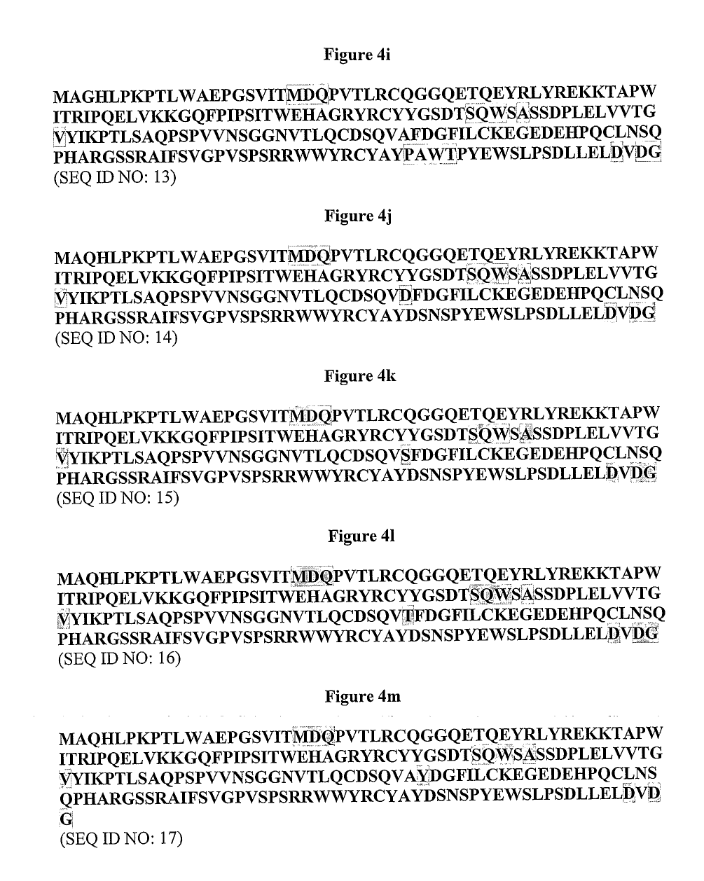

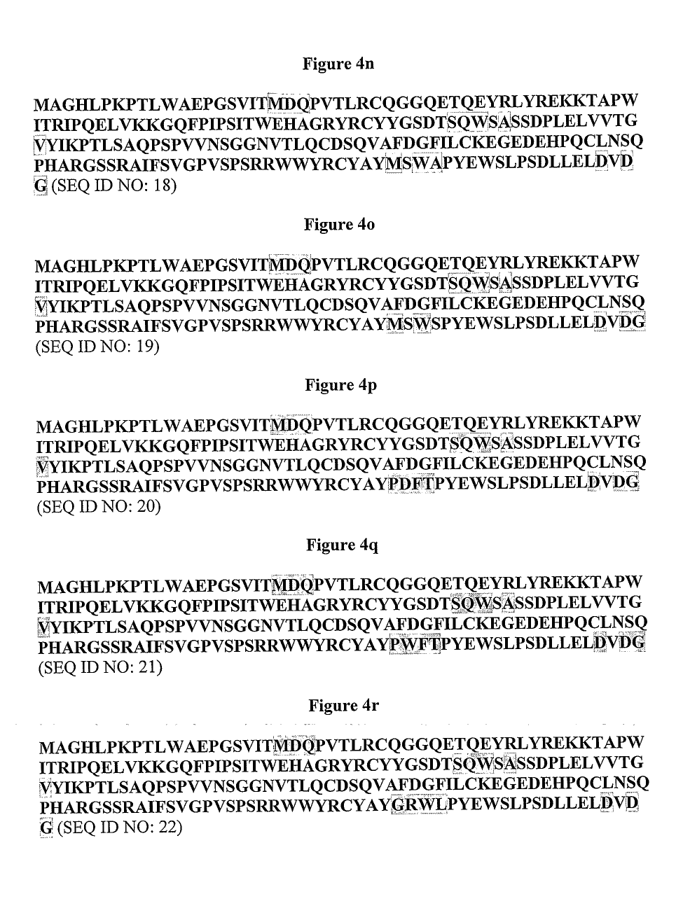

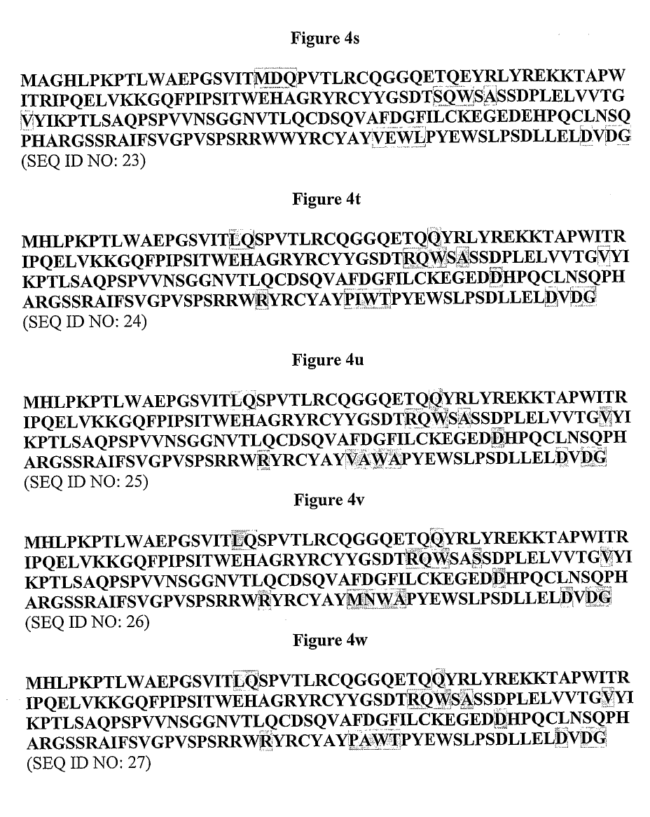

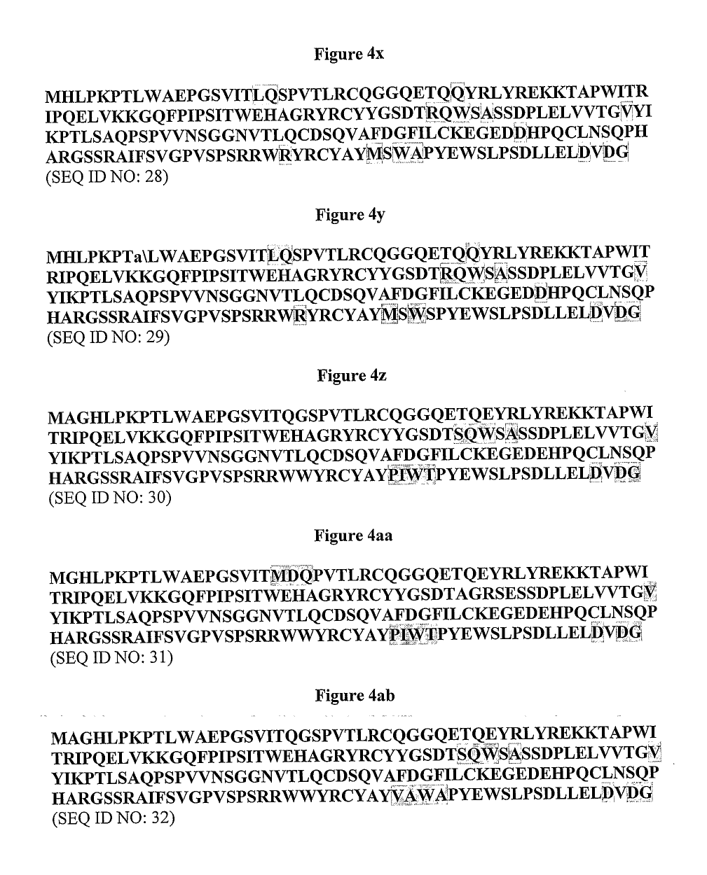

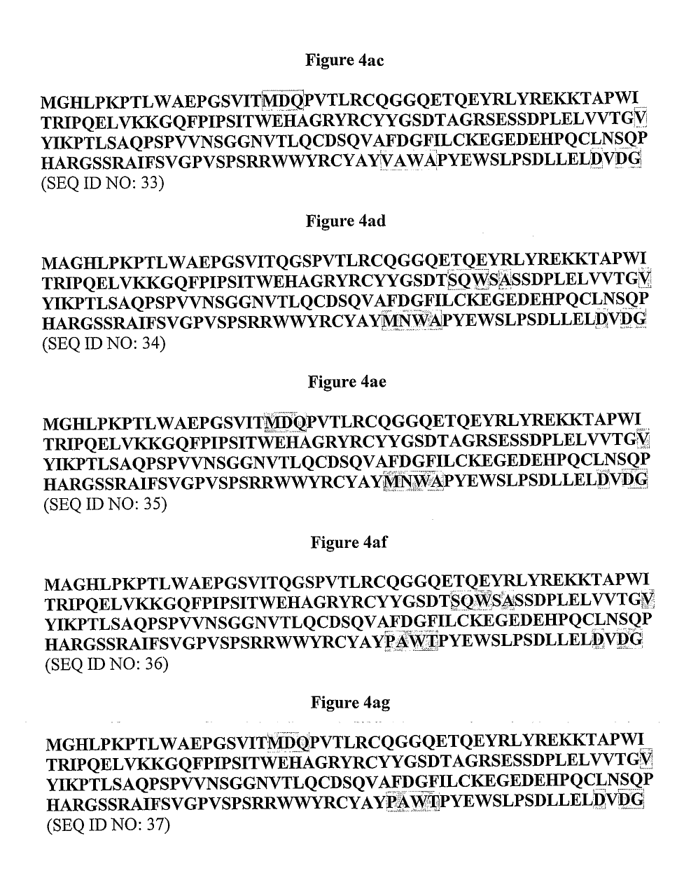

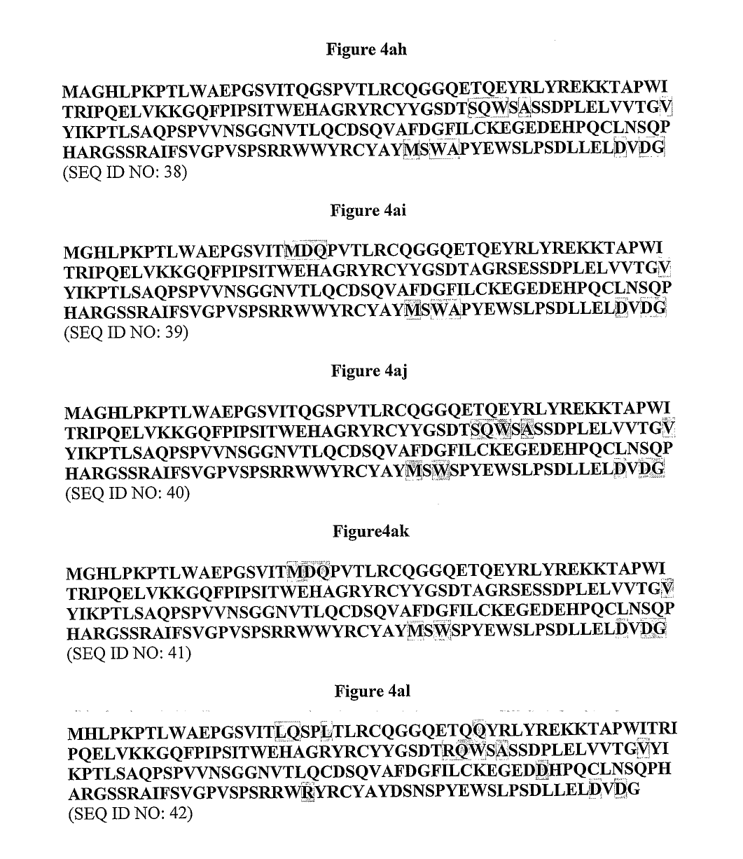

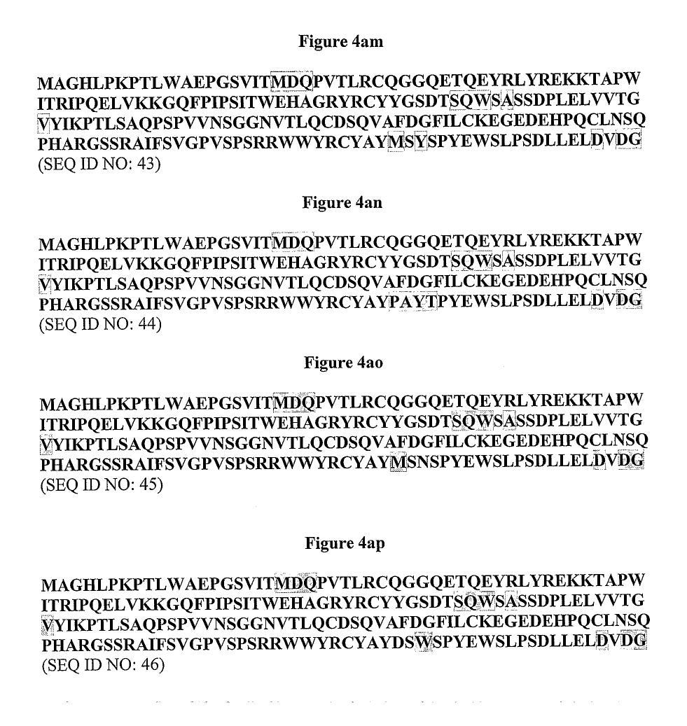

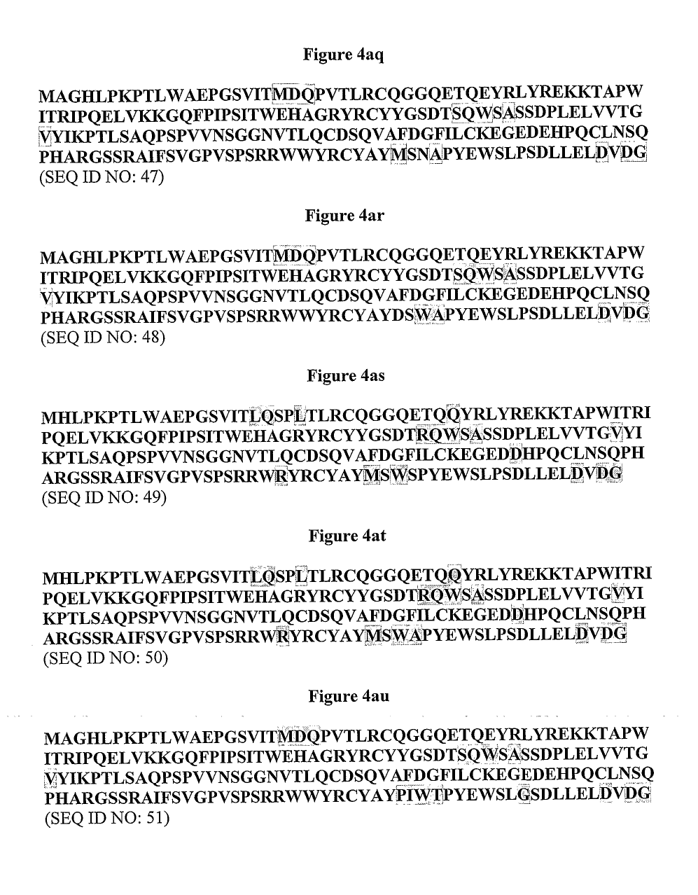

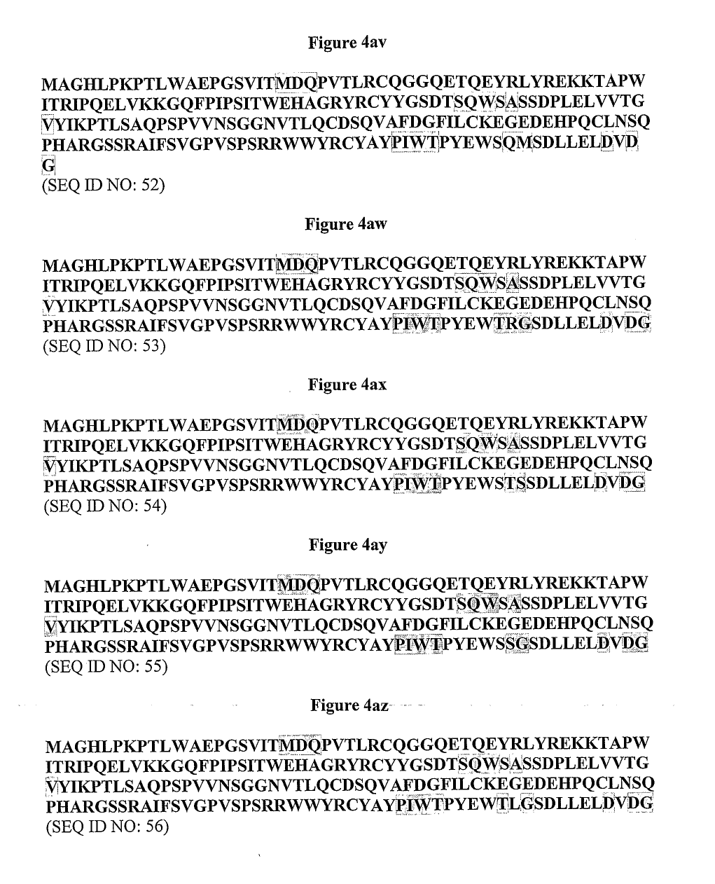

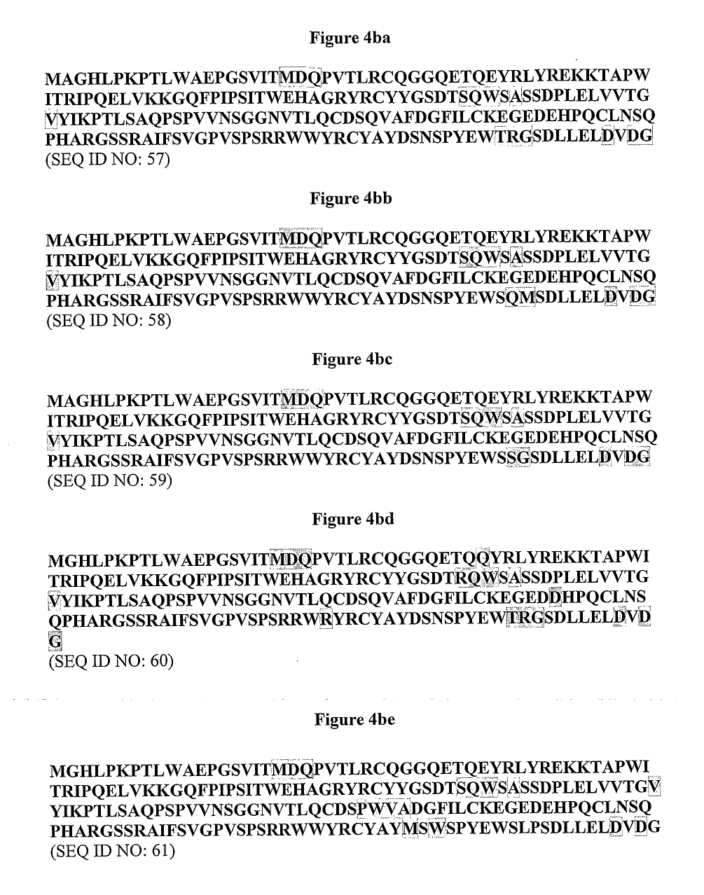

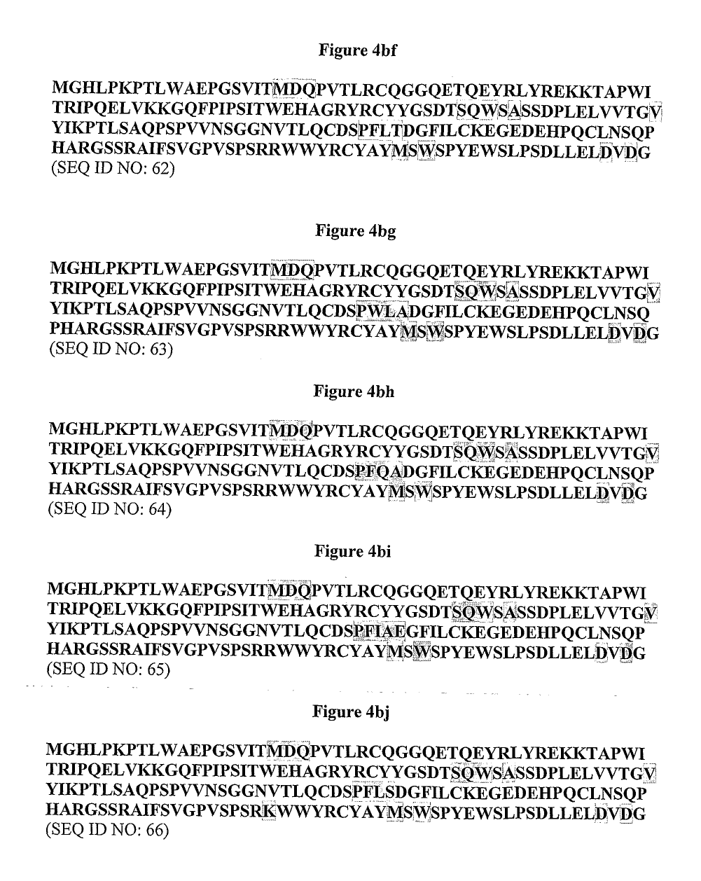

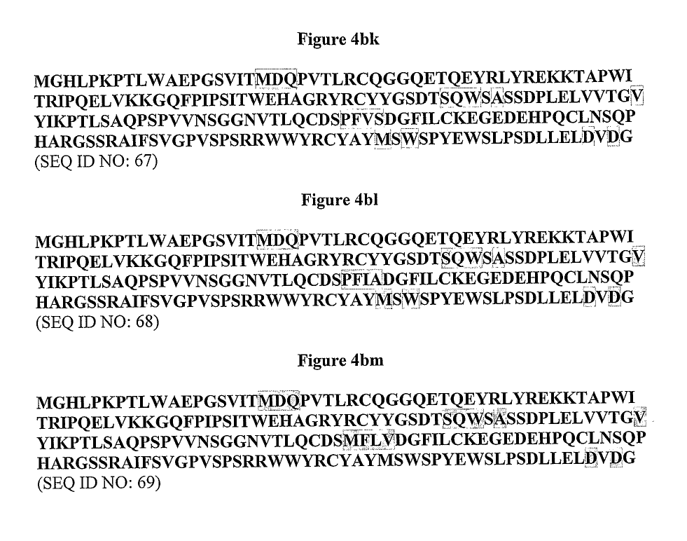

Preferably, such solubility enhancing mutations are in within the C-terminal 6 amino acids of the polypeptides of the invention. The inclusion of one or both of mutations corresponding to 196D and/or 198D using the numbering of SEQ ID NO: 3 provide preferred means of increasing the solubility of the high affinity ILT-like polypeptides of the invention relative to the corresponding polypeptides lacking said mutation(s). The exemplary polypeptides of the invention provided in FIGS. 4a-4bm (SEQ ID NOs: 6 to 69) all incorporate both the 196L→D and 198L→D mutations.

### High Affinity ILT-Like Polypeptides Comprising a C-Terminal Reactive Site

The polypeptides of the invention may be used in multimeric forms or in association with other moieties. In this regard it is desirable to produce polypeptides of the invention which comprising a means of attaching other moieties thereto.

Therefore, one embodiment is provided by a polypeptide of the invention which comprises a C-terminal reactive site for covalent attachment of a desired moiety. This reactive site may be a cysteine residue.

As will be known to those skilled in the art there are many reactive chemistries which are suitable for this purpose. These include, but are not limited to, cysteine residues, hexahistidine peptides, biotin and chemically reactive groups. The presence of such reactive chemistries may also facilitate purification of the polypeptides.

### PEGylated High Affinity ILT-Like Polypeptides

In one particular embodiment a polypeptide of the invention is associated with at least one polyalkylene glycol chain(s). This association may be caused in a number of ways known to those skilled in the art. In a preferred embodiment the polyalkylene chain(s) is/are covalently linked to the polypeptides. In a further embodiment the polyethylene glycol chains of the present aspect of the invention comprise at least two polyethylene repeating units.

### Multivalent High Affinity ILT-Like Complexes

One aspect of the invention provides a multivalent complex comprising at least two polypeptides of the invention, said multivalent complex having a K_{D} for a given Class I pMHC of less than or equal to 1 μM and/or has an off-rate (k_{off}) for the said Class I pMHC of 2 S⁻¹ or slower.

In one embodiment of this aspect, at least two polypeptides of the invention are linked via linker moieties to form multivalent complexes.

One aspect is provided wherein the polypeptides of the invention are linked by a non-peptidic polymer chain or a peptidic linker sequence. Preferably the multivalent complexes are water soluble, so the linker moiety should be selected accordingly. Furthermore, it is preferable that the linker moiety should be capable of attachment to defined positions on the polypeptides, so that the structural diversity of the complexes formed is minimised. One embodiment of the present aspect is provided by a multivalent complex of the invention wherein the polymer chain or peptidic linker sequence extends between amino acid residues of each polypeptide which are not located in the Class I pMHC binding domain of the polypeptides.

Since the complexes of the invention may be for use in medicine, the linker moieties should be chosen with due regard to their pharmaceutical suitability, for example their immunogenicity.

Examples of linker moieties which fulfil the above desirable criteria are known in the art, for example the art of linking antibody fragments.

There are two classes of linker that are preferred for use in the production of multivalent complexes of the present invention. A multivalent complex of the invention in which the polypeptides are linked by a polyalkylene glycol chain or a peptidic linker derived from a human multimerisation domain provide certain embodiments of the invention.

Suitable hydrophilic polymers include, but are not limited to, polyalkylene glycols.

The most commonly used polymers of this class are based on polyethylene glycol or PEG, the structure of which is shown below.

HOCH₂CH₂O(CH₂CH₂O)ₙ—CH₂CH₂OH

Wherein n is greater than two.

However, others are based on other suitable, optionally substituted, polyalkylene glycols include polypropylene glycol, and copolymers of ethylene glycol and propylene glycol.

Such polymers may be used to treat or conjugate therapeutic agents, particularly polypeptide or protein therapeutics, to achieve beneficial changes to the pharmacokinetic (PK) profile of the therapeutic, for example reduced renal clearance, improved plasma half-life, reduced immunogenicity, and improved solubility. Such improvements in the PK profile of the PEG-therapeutic conjugate are believe to result from the PEG molecule or molecules forming a ‘shell’ around the therapeutic which sterically hinders the reaction with the immune system and reduces proteolytic degradation. (Casey et al, (2000) Tumor Targetting 4 235-244) The size of the hydrophilic polymer used may in particular be selected on the basis of the intended therapeutic use of the high affinity ILT-like polypeptides. There are numerous review papers and books that detail the use of PEG and similar molecules in pharmaceutical formulations. For example, see (Harris (1992) Polyethylene Glycol Chemistry—Biotechnical and Biomedical Applications, Plenum, New York, N.Y.) or (Harris & Zalipsky (1997) Chemistry and Biological Applications of Polyethylene Glycol ACS Books, Wash., D.C.).

The polymer used can have a linear or branched conformation. Branched PEG molecules, or derivatives thereof, can be induced by the addition of branching moieties including glycerol and glycerol oligomers, pentaerythritol, sorbitol and lysine.

Usually, the polymer will have a chemically reactive group or groups in its structure, for example at one or both termini, and/or on branches from the backbone, to enable the polymer to link to target sites in the high affinity ILT-like polypeptide. This chemically reactive group or groups may be attached directly to the hydrophilic polymer, or there may be a spacer group/moiety between the hydrophilic polymer and the reactive chemistry as shown below:
Reactive chemistry-Hydrophilic polymer-Reactive chemistry
Reactive chemistry-Spacer-Hydrophilic polymer-Spacer-Reactive chemistry

The spacer used in the formation of constructs of the type outlined above may be any organic moiety that is a non-reactive, chemically stable, chain, Such spacers include, by are not limited to the following:
-(CH₂)ₙ— wherein n=2 to 5
-(CH₂)₃NHCO(CH₂)₂

A multivalent complex of the invention in which a divalent alkylene spacer radical is located between the polyalkylene glycol chain and its point of attachment to a polypeptide molecule of the complex provides a further embodiment of the present aspect.

A multivalent complex of the invention in which the polyalkylene glycol chain comprises at least two polyethylene glycol repeating units provides a further embodiment of the present aspect.

There are a number of commercial suppliers of hydrophilic polymers linked, directly or via a spacer, to reactive chemistries that may be of use in the present invention. These suppliers include Nektar Therapeutics (CA, USA), NOF Corporation (Japan), Sunbio (South Korea) and Enzon Pharmaceuticals (NJ, USA).

Commercially available hydrophilic polymers linked, directly or via a spacer, to reactive chemistries that may be of use in the present invention include, but are not limited to, the following:

| PEG linker | | Catalogue |
|---|---|---|
| Description | Source of PEG | Number |
| ILT-like Monomer attachment | | |
| 5K linear (Maleimide) | Nektar | 2D2MOHO1 |
| 20K linear (Maleimide) | Nektar | 2D2MOPO1 |
| 20K linear (Maleimide) | NOF Corporation | SUNBRIGHT |
| | | ME-200MA |
| 20K branched (Maleimide) | NOF Corporation | SUNBRIGHT |
| | | GL2-200MA |
| 30K linear (Maleimide) | NOF Corporation | SUNBRIGHT |
| | | ME-300MA |
| 40K branched PEG (Maleimide) | Nektar | 2D3XOTO1 |
| 5K-NP linear (for Lys attachment) | NOF Corporation | SUNBRIGHT |
| | | MENP-50H |
| 10K-NP linear (for Lys attachment) | NOF Corporation | SUNBRIGHT |
| | | MENP-10T |
| 20K-NP linear (for Lys attachment) | NOF Corporation | SUNBRIGHT |
| | | MENP-20T |
| ILT-like dimer linkers | | |
| 3.4K linear (Maleimide) | Nektar | 2D2DOFO2 |
| 5K forked (Maleimide) | Nektar | 2D2DOHOF |
| 10K linear (with orthopyridyl ds- | Sunbio | |
| linkers in place of Maleimide) | | |
| 20K forked (Maleimide) | Nektar | 2D2DOPOF |
| 20K linear (Maleimide) | NOF Corporation | |
| 40K forked (Maleimide) | Nektar | 2D3XOTOF |
| Higher order ILT-like multimers | | |
| 15K, 3 arms, Mal₃ (for trimer) | Nektar | OJOONO3 |
| 20K, 4 arms, Mal₄ (for tetramer) | Nektar | OJOOPO4 |
| 40K, 8 arms, Mal₈ (for octamer) | Nektar | OJOOTO8 |

A wide variety of coupling chemistries can be used to couple polymer molecules to protein and peptide therapeutics. The choice of the most appropriate coupling chemistry is largely dependant on the desired coupling site. For example, the following coupling chemistries have been used attached to one or more of the termini of PEG molecules (Source: Nektar Molecular Engineering Catalogue 2003):
N-maleimide
Vinyl sulfone
Benzotriazole carbonate
Succinimidyl proprionate
Succinimidyl butanoate
Thio-ester
Acetaldehydes
Acrylates
Biotin
Primary amines

As stated above non-PEG based polymers also provide suitable linkers for multimerising the polypeptides of the present invention. For example, moieties containing maleimide termini linked by aliphatic chains such as BMH and BMOE (Pierce, products Nos. 22330 and 22323) can be used.

Peptidic linkers are the other class of multivalent complex linkers. These linkers are comprised of chains of amino acids, and function to produce simple linkers or multimerisation domains onto which the polypeptides of the present invention can be attached. The biotin/streptavidin system has previously been used to produce tetramers of TCRs and pMHC molecules (see WO 99/60119) for in-vitro binding studies. However, streptavidin is a microbially-derived polypeptide and as such not ideally suited to use in a therapeutic.

Multivalent complexes of the invention in which the polypeptides are linked by a peptidic linker derived from a human multimerisation domain provide one embodiment of the present aspect. There are a number of human proteins that contain a multimerisation domain that could be used in the production of multivalent high affinity ILT-like polypeptide complexes. For example, the tetramerisation domain of p53 has been utilised to produce tetramers of scFv antibody fragments which exhibited increased serum persistence and significantly reduced off-rate compared to the monomeric scFv fragment. (Willuda et al. (2001) J. Biol. Chem. 276 (17) 14385-14392) Haemoglobin also has a tetramerisation domain that could potentially be used for this kind of application.

In a specific embodiment the multivalent complexes of the invention may be dimers or tetramers. Examples 9 and 10 herein provide detailed methodologies for the production of dimeric and tetrameric PEG-linked high affinity ILT-like complexes of the invention respectively.

A multivalent complex of the invention comprising at least two polypeptides of the invention, wherein at least one of said polypeptide is associated with a therapeutic agent provides a further embodiment of this aspect.

A further aspect is provided by a polypeptide of the invention or multivalent complex thereof wherein said polypeptide or multivalent complex is soluble.

A further aspect is provided by an isolated cell or a particle presenting at least one polypeptide of the invention. As will be obvious to those skilled in the art such polypeptides require a means of attachment to the surface of the said cells or particles. There are a number of means of facilitating such attachment. For example, particularly in the case of cells, this means of attachment may conveniently be provided by producing “full-length” versions of the chosen polypeptides which incorporate at least the transmembrane domain of human ILT-2. The transmembrane domain of human ILT-2 is underlined in FIG. 1a (SEQ ID NO: 1). However, this is not the only means of attaching such polypeptides to the surface of cells. For example, fusions proteins comprising a polypeptide of the invention or fragments thereof linked to the transmembrane domains of other polypeptides may be produced. In the case of attaching the polypeptides of the invention to particles this can conveniently be achieved by contacting polypeptides of the invention which comprise a C-terminal tag, such as biotin, with particles coated with a binding moiety specific for said tag, such as streptavidin.

### Diagnostic and Therapeutic Use

In one aspect the polypeptides of the invention or multivalent complexes thereof may be labelled with an imaging compound, for example a label that is suitable for diagnostic purposes. Such labelled polypeptides are useful in a method for detecting target pMHC molecules which method comprises contacting the pMHC with a polypeptide of the invention or a multivalent complex thereof bind to the pMHC; and detecting said binding. In tetrameric complexes formed for example, using biotinylated polypeptide molecules, fluorescent streptavidin can be used to provide a detectable label. Such a fluorescently-labelled tetramer is suitable for use in FACS analysis, for example to detect antigen presenting cells. Another manner in which the soluble peptides of the present invention may be detected is by the use of antibodies, in particular monoclonal antibodies.

ILT-specific antibodies have been described in the literature. For example, IGH/75 is an ILT-2 specific IgG that was produced at the Basel Institute for Immunology, Basel, Switzerland. (Riteau et al., (2001) Int. Immunol. 13 (2) 193).

In a further aspect a polypeptide of the present invention or a multivalent complex thereof may alternatively or additionally be associated with (e.g. covalently or otherwise linked to) a therapeutic agent or detectable label.

In a specific embodiment of the invention the therapeutic agent is covalently linked to the C terminus of the polypeptide.

There are a number of therapeutic agents which could be associated with the polypeptides of the invention. For example, the therapeutic agent may be an immune effector molecule. A specific embodiment of this aspect is provided wherein the immune effector molecule is a cytokine. As is known to those skilled in the art there are a number of cytokines which generally act to “suppress” immune responses. Polypeptides of the invention associated with such immuno-suppressive cytokines form preferred embodiments of the invention. Polypeptides of the invention associated with IL-4, IL-10 or IL-13 or a phenotypically silent variant or fragment of these cytokines provide specific embodiments of the present invention.

A multivalent complex of the invention may have enhanced binding capability for a given pMHC compared to a non-multimeric wild-type ILT or the corresponding high affinity ILT-like polypeptide of the invention. Thus, the multivalent complexes according to the invention are particularly useful for tracking or targeting cells presenting particular antigens in vitro or in vivo, and are also useful as intermediates for the production of further multivalent complexes having such uses.

Pharmaceutical compositions comprising a polypeptide of the invention, or a multivalent complex thereof, or a plurality of cells expressing such polypeptides, together with a pharmaceutically acceptable carrier therefore provide a further aspect of the invention. A related embodiment is provided by the therapeutic use of a polypeptide of the invention, or a multivalent complex thereof, or a plurality of cells expressing such polypeptides.

Pharmaceutical compositions comprising a polypeptide of the invention or a multivalent complex thereof associated with a therapeutic agent together with a pharmaceutically acceptable carrier therefore provide a further aspect of the invention. A related embodiment is provided by the therapeutic use of a polypeptide of the invention or a multivalent complex thereof associated with a therapeutic agent.

Another aspect of the invention is provided by the use of a polypeptide of the invention or a multivalent complex thereof in the manufacture of a medicament for the treatment of autoimmune disease. In certain embodiments of the present aspect said medicament may be adapted for parenteral administration. Suitable parenteral routes of administration include subcutaneous, intradermal or intramuscular routes.

A further aspect of the inventions is provided by the use of a polypeptide of the invention or a multivalent complex thereof associated with a therapeutic agent, in the manufacture of a medicament for the treatment of autoimmune disease, In certain embodiments of the present aspect said medicament may be adapted for parenteral administration. Suitable parenteral routes of administration include subcutaneous, intradermal or intramuscular routes.

Autoimmune diseases which may be amenable to treatment by the compostions of the present invention include, but are not limited to, Asthma, Diabetes, Goodpasture's syndrome, Multiple sclerosis, Psoriasis and Rheumatoid arthritis,

Soluble polypeptide of the invention or a multivalent complex thereof of the invention may be linked to an enzyme capable of converting a prodrug to a drug. This allows the prodrug to be converted to the drug only at the site where it is required (i.e. targeted by the polypeptide of the invention or a multivalent complex thereof).

It is expected that the polypeptide of the invention or a multivalent complex thereof f disclosed herein may be used in methods for the diagnosis and treatment of autoimmune disease.

The invention also provides a method of treatment of autoimmune disease comprising administering to a subject suffering such autoimmune disease an effective amount of a polypeptide of the invention or multivalent complex thereof, or a plurality of cells or particles presenting at least one such polypeptide. In a related embodiment the invention provides for the use of a polypeptide of the invention or multivalent complex thereof, or a plurality of cells or particles presenting at least one such polypeptide, in the preparation of a composition for the treatment of autoimmune disease. Example 11 herein provides in-vitro data on the ability of a high affinity ILT-like polypeptide of the invention, specifically the c83 polypeptide (SEQ ID NO: 19), to inhibit cytotoxic T cell activation.

The invention also provides a method of treatment of autoimmune disease comprising administering to a subject suffering such autoimmune disease an effective amount of a polypeptide of the invention or a multivalent complex thereof associated with a therapeutic agent. In a related embodiment the invention provides for the use of a polypeptide of the invention or a multivalent complex thereof, associated with a therapeutic agent, in the preparation of a composition for the treatment of autoimmune disease.

Therapeutic or imaging polypeptides in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example parenteral, transdermal or via inhalation, preferably a parenteral (including subcutaneous, intramuscular, or, most preferably intravenous) route. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. and a physician will ultimately determine appropriate dosages to be used.

### Additional Aspects

A polypeptide or multivalent complex of the present invention may be provided in substantially pure form, or as a purified or isolated preparation. For example, it may be provided in a form which is substantially free of other proteins.

Further embodiments are provided by an isolated nucleic acid encoding the polypeptides of the invention, vectors incorporating said nucleic acid and cells containing said vectors. The nucleic acid encoding the polypeptide of the invention may be one which has been adapted for high level expression in a host cell. There are a number of companies which offer such nucleic acid optimisation as a service, for example GeneArt AG, Germany.

A final aspect is provided by a method of producing a polypeptide of the invention comprising:
(i) transforming a host cell with a vector incorporating nucleic acid encoding a polypeptide of the invention; and
(ii) culturing the transformed cells under conditions suitable for the expression of a polypeptide of the invention; and
(iii) recovering the expressed polypeptide.

Specific embodiments of the present aspect are provided wherein the host cells are E. coli cells or yeast cells, for example Pichia pastoris cells. Examples 1 to 3, and 7 to 8 herein provide detailed methodologies for the production of polypeptides of the invention in E. coli and Pichia Pastoris cells respectively.

Preferred features of each aspect of the invention are as for each of the other aspects mutatis mutandis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

FIG. 1a is the full amino acid sequence of a wild type human ILT-2. (SEQ ID No: 1) The highlighted amino acids show residues of this polypeptide which differ from the corresponding residues of isoform 1 of Wild-type human ILT-2. The amino acids of the transmembrane domain are underlined.

FIG. 1b is the full DNA sequence of a wild type human ILT-2 (SEQ ID No: 2) which encodes the amino acid sequence of FIG. 1a. The DNA sequence corresponds to that given NCIMB Nucleotide accession NO: NM_{—}006669.

FIGS. 2a and 2b respectively are the amino acid and DNA sequence of a soluble two domain form of the wild-type ILT-2 sequences provided in FIGS. 1a and 1b. These truncated sequences contain/encode for only extracellular domains D1 and D2 of ILT-2. (SEQ ID No: 3 and SEQ ID NO: 4 respectively)

FIG. 3 is the full DNA sequence inserted into the pGMT7-based vector in order to express the soluble two domain form of the wild-type ILT-2 polypeptide of FIG. 2a. The HindIII and NdeI restriction enzyme recognition sequences are underlined.

FIGS. 4a to 4bm (SEQ ID Nos 6-69) are the amino acid sequences of soluble two domain high affinity ILT-like polypeptides. The residues which have been mutated relative to those of FIG. 2a are highlighted

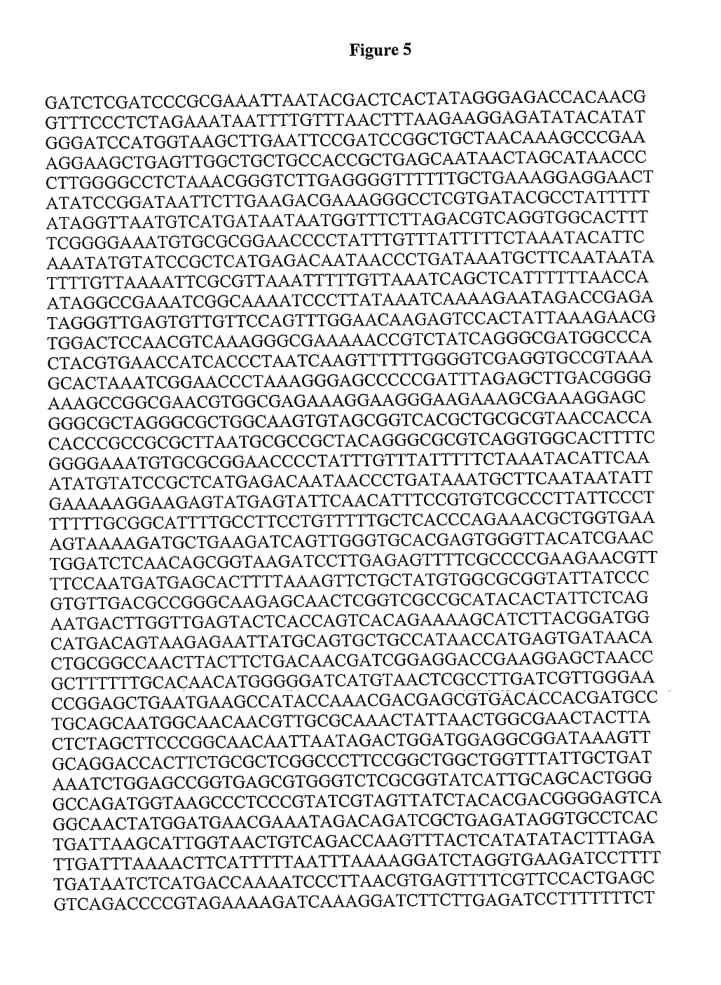

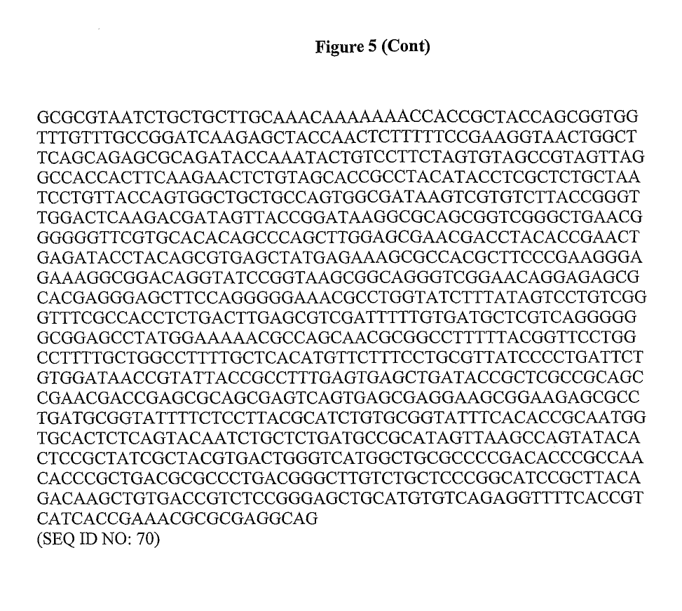

FIG. 5 is the DNA sequence of a pGMT7-derived vector into which DNA encoding the amino acid sequences of FIGS. 4a to 4bm can be inserted.

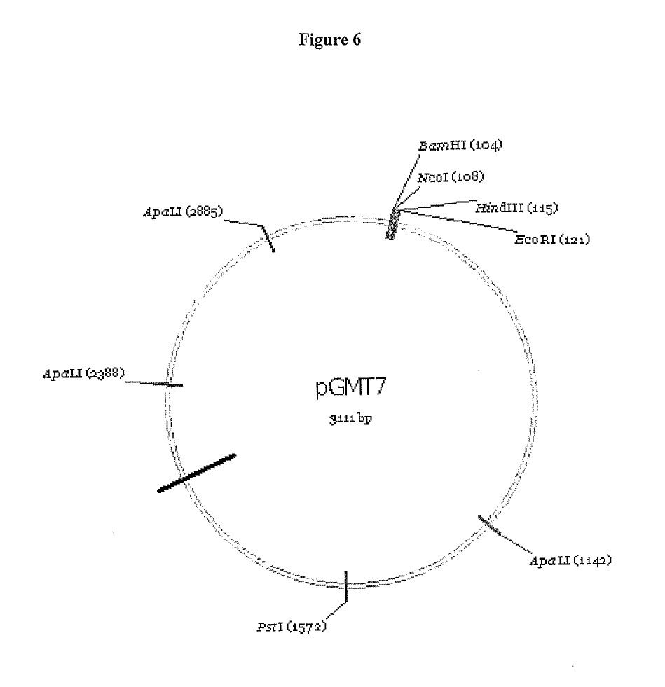

FIG. 6 is the plasmid map of a pGMT7-derived vector into which the DNA sequences encoding the amino acid sequences of the high affinity ILT-like polypeptides of FIGS. 4a to 4bm can be inserted.

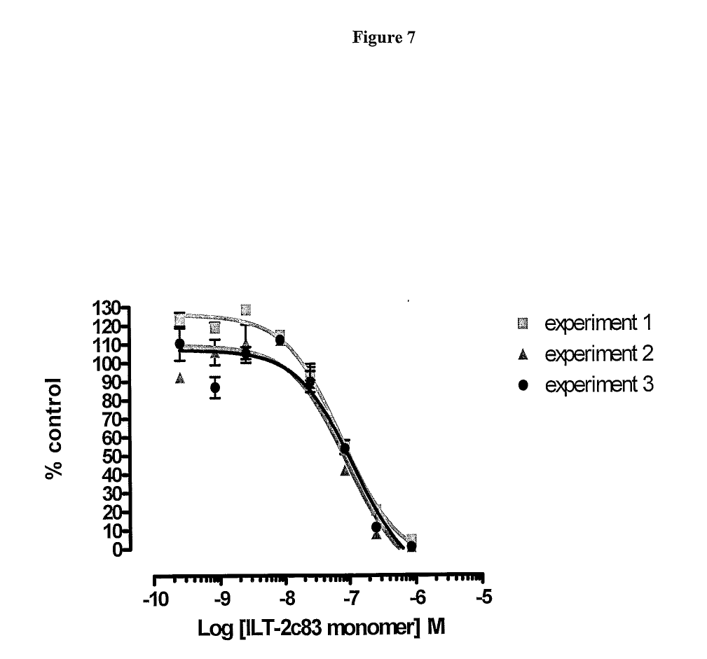

FIG. 7 is a graph showing the results of three ELISPOT determinations of the ability of the clone 83 (c83) ILT-2 polypeptide to inhibit CTL activation. (See SEQ ID NO: 19 for the full amino acid sequence of this polypeptide)

### Example 1

### Production of a Soluble Wild-Type ILT-2 Molecule Comprising Domains 1 and 2

FIG. 3 (SEQ ID NO: 5) provides the DNA sequence used to express a soluble wild-type ILT-2 containing only domains D1 and D2. This DNA sequence was synthesised de-novo by a contract research companies, GeneArt AG (Germany). Restriction enzyme recognition sites (NdeI and HindIII) have been introduced into this DNA sequence in order to facilitate ligation of the DNA sequence into a pGMT7-based expression plasmid, which contains the T7 promoter for high level expression in E. coli strain BL21-DE3(pLysS) (Pan et al., Biotechniques (2000) 29 (6): 1234-8)

This DNA sequence is ligated into a pGMT7 vector cut with NdeI and HindIII. (See FIG. 5 for the DNA sequence of this vector and FIG. 6 for the plasmid map of this vector).

Restriction Enzyme Recognition Sites as Introduced into DNA Encoding the Soluble Wild-Type ILT-2 Polypeptide

NdeI-CATATG
HindIII-AAGCTT

### Ligation

The cut ILT-2 DNA and cut vector are ligated using a rapid DNA ligation kit (Roche) following the manufacturers instructions.

Ligated plasmids are transformed into competent E. coli strain XL1-blue cells and plated out on LB/agar plates containing 100 mg/ml ampicillin. Following incubation overnight at 37° C., single colonies are picked and grown in 10 ml LB containing 100 mg/ml ampicillin overnight at 37° C. with shaking. Cloned plasmids are purified using a Miniprep kit (Qiagen) and the insert is sequenced using an automated DNA sequencer (Lark Technologies).

FIG. 2a shows the amino acid sequence of the soluble wild-type ILT-2 polypeptide produced from the DNA sequence of FIG. 2b.

### Example 2

### Production of High Affinity Variants of the Soluble Wild-Type ILT-2 Polypeptide

The soluble wild-type ILT-2 polypeptide produced as described in Example 1 can be used a template from which to produce the polypeptides of the invention which have an increased affinity and/or slower off-rate for class I pMHC molecules.

As is known to those skilled in the art the necessary codon changes required to produce these mutated chains can be introduced into the DNA encoding the soluble wild-type ILT-2 polypeptide by site-directed mutagenesis. (QuickChange™ Site-Directed Mutagenesis Kit from Stratagene)

Briefly, this is achieved by using primers that incorporate the desired codon change(s) and the plasmids containing the DNA encoding the soluble wild-type ILT-2 polypeptide as a template for the mutagenesis:

Mutagenesis was carried out using the following conditions: 50 ng plasmid template, 1 μl of 10 mM dNTP, 5 μl of 10×Pfu DNA polymerase buffer as supplied by the manufacturer, 25 μmol of fwd primer, 25 μmol of rev primer, 1 μl pfu DNA polymerase in total volume 50 μl. After an initial denaturation step of 2 mins at 95 C, the reaction was subjected to 25 cycles of denaturation (95 C, 10 secs), annealing (55 C 10 secs), and elongation (72 C, 8 mins). The resulting product was digested with DpnI restriction enzyme to remove the template plasmid and transformed into E. coli strain XL1-blue. Mutagenesis was verified by sequencing.

The amino sequences of the mutated ILT-like polypeptides which demonstrate high affinity for the YLSGANLNL (SEQ ID NO: 71)-HLA-A*0201 complex are listed in FIGS. 4a to 4bm (SEQ ID Nos: 6 to 69). As is known to those skilled in the art the necessary codon changes required to produce these mutated polypeptides can be introduced into the DNA encoding the wild-type soluble ILT-2 polypeptide by site-directed mutagenesis. (QuickChange™ Site-Directed Mutagenesis Kit from Stratagene)

### Example 3

### Expression, Refolding and Purification of Soluble Polypeptides

The expression plasmid containing the ILT polypeptides as prepared in Examples 1 or 2 are transformed separately into E. coli strain rosetta DE3pLysS, and single ampicillin/chloramphenicol-resistant colonies are grown at 37° C. in TYP (ampicillin 100 μg/ml, chloramphenicol 15 μg/ml) medium for 7 hours before inducing protein expression with 0.5 mM IPTG. Cells are harvested 15 hours post-induction by centrifugation for 30 minutes at 4000 rpm in a Beckman J-6B. Cell pellets are re-suspended in a buffer, re-suspended cells are sonicated in 1 minute bursts for a total of around 10 minutes in a Milsonix XL2020 sonicator using a standard 12 mm diameter probe. Inclusion body pellets are recovered by centrifugation for 10 minutes at 4000 rpm in a Beckman J2-21 centrifuge. Three detergent washes are then carried out to remove cell debris and membrane components. Each time the inclusion body pellet is homogenised in a Triton buffer (50 mM Tris-HCl, 0.5% Triton-X100, 200 mM NaCI, 10 mM NaEDTA, 0.1% (w/v) NaAzide, 2 mM DTT, pH 8.0) before being pelleted by centrifugation for 15 minutes at 4000 rpm in a Beckman J2-21. Detergent and salt is then removed by a similar wash in the following buffer: 50 mM Tris-HCl, 1 mM NaEDTA, 0.1% (w/v) NaAzide, 2 mM DTT, pH 8.0. Finally, the inclusion bodies are divided into 60 mg aliquots and frozen at −70° C. Inclusion body protein yield is quantitated by solubilising with 6M guanidine-HCl and measurement using a UV spectrometer.

Approximately 60 mg of ILT polypeptide solubilised inclusion bodies are thawed from frozen stocks and diluted into 15 ml of a guanidine solution (6 M Guanidine-hydrochloride, 10 mM Sodium Acetatae, 10 mM EDTA), to ensure complete chain denaturation. The guanidine solution containing fully reduced and denatured ILT polypeptide is then injected into 1 litre of the following refolding buffer: 100 mM Tris pH 8.5, 400 mM L-Arginine, 2 mM EDTA, 5 mM reduced Cystaeimine, 0.5 mM 2-mercaptoethylamine, 5M urea. The redox couple (2-mercaptoethylamine and cystamine (to final concentrations of 6.6 mM and 3.7 mM, respectively) are added approximately 5 minutes before addition of the denatured ILT polypeptide. The solution is left for 30 minutes. The refolded ILT polypeptide was dialysed in Spectrapor 1 membrane (Spectrum; Product No. 132670) against 10 L 10 mM Tris pH 8.1 at 5° C.±3° C. for 18-20 hours. After this time, the dialysis buffer is changed to fresh 10 mM Tris pH 8.1 (10 L) and dialysis is continued at 5° C.±3° C. for another 20-22 hours.

Soluble ILT polypeptide is separated from degradation products and impurities by loading the dialysed refold onto a POROS 50HQ anion exchange column and eluting bound protein with a gradient of 0-500 mM NaCI over 50 column volumes using an Akta purifier (Pharmacia). Peak fractions are stored at 4° C. and analysed by Coomassie-stained SDS-PAGE before being pooled and concentrated. Finally, the soluble ILT polypeptide is purified and characterised using a Superdex 200HR gel filtration column pre-equilibrated in HBS-EP buffer (10 mM HEPES pH 7.4, 150 mM NaCl, 3.5 mM EDTA, 0.05% nonidet p40). The peak eluting at a relative molecular weight of approximately 27 kDa is pooled and concentrated prior to characterisation by Biacore surface plasmon resonance analysis.

### Example 4

### Biacore Surface Plasmon Resonance Characterisation of the Binding of Soluble ILT Molecules to pMHC Molecules

A surface plasmon resonance biosensor (BiaCore 3000™) was used to analyse the binding of soluble ILT molecules to class I pMHC. This was facilitated by producing soluble biotinylated pMHC (described below) which were immobilised to a streptavidin-coated binding surface in a semi-oriented fashion, allowing efficient testing of the binding of a soluble ILT molecule to up to four different pMHC (immobilised on separate flow cells) simultaneously. Injection of the pMHC allows the precise level of immobilised class I molecules to be manipulated easily.

Soluble biotinylated class I HLA-A*0201 loaded with a CEA-derived YLSGANLNL (SEQ ID NO: 71) peptide were refolded in vitro from bacterially-expressed inclusion bodies containing the constituent subunit proteins and synthetic peptide, followed by purification and in vitro enzymatic biotinylation (O'Callaghan et al. (1999) Anal. Biochem. 266: 9-15). MHC-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The MHC light-chain or β2-microglobulin was also expressed as inclusion bodies in E. coli from an appropriate construct, at a level of ˜500 mg/litre bacterial culture.

The E. coli cells were lysed and inclusion bodies are purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2m into 0.4 M L-Arginine-HCl, 100 mM Tris pH 8.1, 3.7 mM cystamine, 6.6 mM β-cysteamine, 4 mg/ml of the peptide required to be loaded by the MHC, by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. Two changes of buffer were necessary to reduce the ionic strength of the solution sufficiently. The protein solution was then filtered through a 1.5 μm cellulose acetate filter and loaded onto a POROS 50HQ anion exchange column (8 ml bed volume). Protein was eluted with a linear 0-500 mM NaCl gradient. The soluble biotinylated HLA-A2-peptide complex eluted at approximately 250 mM NaCl, and peak fractions were collected, a cocktail of protease inhibitors (Calbiochem) was added and the fractions were chilled on ice.

Biotinylation tagged pMHC were buffer exchanged into 10 mM Tris pH 8.1, 5 mM NaCl using a Pharmacia fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl2, and 5 μg/ml BirA enzyme (purified according to O'Callaghan et al. (1999) Anal. Biochem. 266: 9-15). The mixture was then allowed to incubate at room temperature overnight.

Biotinylated pMHC were purified using gel filtration chromatography. A Pharmacia Superdex 75 HR 10/30 column was pre-equilibrated with filtered PBS and 1 ml of the biotinylation reaction mixture was loaded and the column was developed with PBS at 0.5 ml/min. Biotinylated pMHC eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated pMHC were stored frozen at −20° C. Streptavidin was immobilised by standard amine coupling methods.

Such immobilised pMHC are capable of binding soluble T-cell receptors and the co-receptor CD8αα, as well as ILT molecules, and these interactions can be used to ensure that the immobilised pMHC are correctly refolded.

The interactions between a soluble ILT molecule and CEA-derived YLSGANLNL (SEQ ID NO: 71)-HLA-A*0201, the production of which is described above, were analysed on a Biacore 3000™ surface plasmon resonance (SPR) biosensor. SPR measures changes in refractive index expressed in response units (RU) near a sensor surface within a small flow cell, a principle that can be used to detect receptor ligand interactions and to analyse their affinity and kinetic parameters. The probe flow cells were prepared by immobilising the pMHC complexes in flow cells via biotin-tag binding. The assay was then performed by passing soluble ILT over the surfaces of the different flow cells at a constant flow rate, measuring the SPR response in doing so.

### To Measure Equilibrium Binding Constant

Serial dilutions of soluble ILT molecules were prepared and injected at constant flow rate of 5 μl min-1 over two different flow cells; one coated with ˜500 RU of the specific-HLA-A*0201 complex, the second cell was left blank as a control. Response was normalised for each concentration using the measurement from the control cell. Normalised data response was plotted versus concentration of ILT sample and fitted to a hyperbola in order to calculate the equilibrium binding constant, K_{D}. (Price & Dwek, Principles and Problems in Physical Chemistry for Biochemists (2^{nd} Edition) 1979, Clarendon Press, Oxford).

### To Measure Kinetic Parameters

For high affinity soluble ILTs K_{D} was determined by experimentally measuring the dissociation rate constant, kd, and the association rate constant, ka. The equilibrium constant K_{D} was calculated as kd/ka.

High affinity ILT-like molecules were injected over two different cells one coated with ˜300 RU of CEA-derived YLSGANLNL (SEQ ID NO: 71)-HLA-A*0201 complex, the second was left blank as a control Flow rate was set at 50 μl/min. Typically 250 μl of ILT polypeptide at ˜3 μM was injected. Buffer was then flowed over until the response had returned to baseline. Kinetic parameters were calculated using Biaevaluation software. The dissociation phase was also fitted to a single exponential decay equation enabling calculation of half-life.

### Results

The interaction between a soluble variant of wild-type ILT-2 and the CEA-derived YLSGANLNL (SEQ ID NO: 71)-HLA-A*0201 complex was analysed using the above methods and demonstrated a K_{D} of approximately 6 μM. The ILT-like molecules having the amino acid sequences provided in FIGS. 4a to 4bm (SEQ ID Nos: 6 to 69 have a K_{D} of less than or equal to 1 μM and/of 2 S⁻¹ or slower.

### Example 5

### Biacore Surface Plasmon Resonance Analysis of Soluble ILT-Mediated Inhibition of the pMHC/CD8 Interaction

A surface plasmon resonance biosensor (Biacore 3000™) is used to analyse soluble ILT-mediated inhibition of the pMHC/CD8 interaction. This is facilitated by producing soluble pMHC complexes (described below) and biotinylated soluble CD8αα molecules (also described below). The biotinylated soluble CD8αα molecules are immobilised to a streptavidin-coated binding surface “Biacore chip” in a semi-oriented fashion, allowing efficient testing of the binding of soluble pMHC complexes to the immobilised soluble CD8αα. Injection of the biotinylated soluble CD8αα molecules allows the precise level of immobilised CD8 molecules to be manipulated easily.

Soluble HLA-A*0201 pMHC loaded with a CEA-derived YLSGANLNL (SEQ ID NO: 71) peptide are produced using the methods substantially as described in (Garboczi et. al., (1992) PNAS USA 89 3429-3433). The soluble pMHC molecules are refolded in vitro from E. coli expressed inclusion bodies containing the constituent subunit proteins and synthetic peptide and then purified. The MHC light-chain or 132-microglobulin is also expressed as inclusion bodies in E. coli from an appropriate construct, at a level of ˜500 mg/litre bacterial culture.

E. coli cells are lysed and inclusion bodies are purified, and the over-expressed proteins are refolded and purified using the methods detailed in Example 4 except that the biotinylation steps are omitted.

Biotinylated soluble CD8 molecules are produced as described in Examples 1 and 6 of EP1024822. Briefly, the soluble CD8α containing a C-terminal biotinylation tag is expressed as inclusion bodies in E. coli and then purified and refolded to produce CD8αα homodimers containing a tag sequence that can be enzymatically biotinylated. (Schatz, (1993) Biotechnology NY 11: 1138-43). Biotinylation of the tagged CD8α molecules is then achieved using the BirA enzyme (O'Callaghan, et al. Anal Biochem 266(1): 9-15 (1999) Biotinylation reagents are: 1 mM biotin, 5 mM ATP (buffered to pH 8), 7.5 mM MgCl2, and 5 μg/ml BirA enzyme (purified according to O'Callaghan et al. (1999) Anal. Biochem. 266: 9-15). The mixture is then allowed to incubate at room temperature overnight.

The biotinylated sCD8αα is immobilised on the surface of a Biacore streptavidin-coated chip producing a change in the refractive index of 1000 response units. Such immobilised CD8αα molecules are capable of binding soluble pMHC complexes which may be injected in the soluble phase.

The ability of the ILT molecules to inhibit the pMHC/CD8 interaction on a Biacore 3000™ surface plasmon resonance (SPR) biosensor is analysed as follows:

SPR measures changes in refractive index expressed in response units (RU) near a sensor surface within a small flow cell, a principle that can be used to detect receptor ligand interactions and to analyse their affinity and kinetic parameters. The chips are prepared by immobilising the soluble biotinylated CD8αα molecules to streptavidin coated chips as described above. Serial dilutions of soluble wild-type ILT-2 (SEQ ID NO: 3) or high affinity ILT-like molecules are prepared and injected at constant flow rate of 5 μl min⁻¹ over a flow cell coated with 1000 RU of biotinylated CD8αα in the presence of a suitable concentration of soluble YLSGANLNL (SEQ ID NO: 71)-HLA-A*0201 The inhibition of the SPR responses for the CD8αα/pMHC interaction produce a dose response curve which is used to calculate an IC50 value for the polypeptide being assayed for this interaction.

### Example 6

### Comparison of Polypeptide Sequence Identity and Similarity

The protein-protein comparison algorithm used to generate identity and similarity data for this application is available via the following website:

http://fasta.bioch.virginia.edu/fasta_www/cgi/search_frm2.cgi

The “FASTA: protein: protein DNA: DNA” programme available on this website was used to carry out these comparisons. The following (default) settings were used:

### Ktup: Ktup=2

Scoring matrix: Blosum 50

### Gap: −10

### Ext: −2

In order to run the required comparisons the soluble ILT-2 fragment amino acid sequence in single letter code as provided in FIG. 4o (SEQ ID NO: 19) is entered as the first (query) sequence and the amino acid sequence for comparison thereto is entered as the second (library) sequence. The algorithm can then be run and will provide percentage identity and similarity scores for the pair of sequences compared.

As will be obvious to those skilled in the art there are a number of sources of FASTA protein: protein comparisons which could be used for this analysis.

### Example 7

### Production of Pichia pastoris Vectors for the Expression of Soluble High Affinity ILT-Like Polypeptide

DNA encoding any of the high affinity soluble high affinity ILT-like polypeptides containing only domains D1 and D2 shown in FIG. 4a-4bm can be optimised for expression in Pichia pastoris. This DNA sequence optimisation can be synthesised de-novo by a number of contract research companies, for example GeneArt AG (Germany). A cysteine-encoding codon can be added to the 3′ prime end of this DNA in order to provide a “tag” on the C-terminus of the expressed ILT-like polypeptide to facilitate multimerisation if required. Restriction enzyme recognition sites (SnaBI and NotI) can be introduced into this DNA sequence in order to facilitate ligation of the DNA sequence into a pPIC9K expression plasmid. (Invitrogen)

Restriction Enzyme Recognition Sites as Introduced into DNA Encoding the Soluble High Affinity ILT-Like Polypeptides:

SnaBI-tacgta
NotI-gcggccgc

### Ligation

The high affinity ILT-like polypeptide-encoding DNA sequence can be ligated into a pPIC9K vector (Invitrogen) cut with SnaBI and NotI restriction enzymes using a rapid DNA ligation kit (Roche).

### Plasmid Amplification

Ligated plasmids are transformed into competent XL1 blue, (Stratagene, Country) and plated out on LB/agar plates containing 100 mg/ml Kanamycin. Following incubation overnight at 37° C., single colonies are picked and grown in 100 ml LB containing 100 mg/ml Kanamycin overnight at 37° C. with shaking. Cloned plasmids are purified using a Midiprep kit (Qiagen) and the insert is sequenced using an automated DNA sequencer (Lark Technologies).

### Example 8

### Expression and Purification of Soluble High Affinity ILT-Like Polypeptides in Pichia pastoris

The Pichia pastoris expression plasmid containing the affinity ILT-like polypeptide-encoding DNA as prepared in Example 7 are transformed into Pichia pastoris strain GS115 (Invitrogen, USA) as follows;

GS115 Pichia pastoris cells are made competent using a Pichia EasyComp Kit (Invitrogen). This kit uses PEG1000 to make the cells chemically competent.

The ILT-like polypeptide DNA containing vector is linearised using Sal I and transformed into the GS115 strain as described in the Invitrogen manual.

Transformants containing the high affinity ILT-like polypeptide encoding DNA are selected by growing cells on RDB agar plates (Invitrogen). The RDB agar lacks histidine, ensuring that only yeast cells which have been successfully transformed with the pPIC9K plasmid will grow. The pPIC9K plasmid imparts the ability of growing on histidine agar by providing a copy of the HIS4 gene which allows growth on His-media.

Single colonies are picked from the agar plate and grown at 30° C. in BMGY medium (Invitrogen) overnight before inducing protein expression. Protein expression is induced by spinning the cells (2000×g, 10 min) and resuspending in 200 ml BMMY induction media (Invitrogen). Cells are harvested 6 days post-induction by centrifugation for 30 minutes at 2000×g. The supernatant is concentrated down via tangential flow filtration (Sartorious 10 kDa cut off) to 10 ml and purified using SEC (S200HR GE Healthcare)

Peak fractions are stored at 4° C. and analysed by Coomassie-stained SDS-PAGE before being pooled and concentrated. Finally, the soluble high affinity ILT-like polypeptide is purified and characterised using a Superdex 200HR gel filtration column pre-equilibrated in HBS-EP buffer (10 mM HEPES pH 7.4, 150 mM NaCl, 3.5 mM EDTA, 0.05% nonidet p40). The peak eluting at a relative molecular weight of approximately 27 kDa is pooled and concentrated prior to characterisation by Biacore surface plasmon resonance analysis using the methods detailed in Example 4.

### Example 9

### Dimerisation of ILT-Like Polypeptides Using a 3.4 kd Mal-PEG-Mal Linker

Soluble High affinity ILT-like polypeptides containing an additional cysteine residue at the C-terminus are prepared using the methods detailed in Examples 1 to 3. The ILT-like polypeptides are cross-linked using non-branched bifunctional maleimide-PEG (MAL-PEG-MAL, MW 3.4 KD, NOF Corporation, Japan). The maleimide groups on the termini of this linker confer free thiol binding specificity to the linker. Prior to cross-linking the ILT-like polypeptide is pre-treated with a reducing agent, 0.1 mM DTT (room temperature, overnight), in order to liberate the free cysteine on the soluble ILT-like polypeptide. This low concentration of reducing agent is used to selectively reduce the exposed C-terminal cysteine residue. The soluble ILT-like polypeptide is then re-purified by gel-filtration chromatography (Superdex 75) in PBS buffer. The ILT-like polypeptide is then re-concentrated using a 10 kDa cut-off centrifugal membrane concentrator (VivaScience, Satorius). Cross-linking is achieved by the stepwise addition of MAL-PEG-MAL (10 mM in DMF) at an approximately 2:1 (protein to cross-linker) molar ratio and subsequently incubating for 2 hours at room temperature. The product is then purified using Superdex 75 HR10/30 gel-filtration column pre-equilibrated in PBS. Three peaks are observed after cross-linking; of which one corresponded with the position of intact “monomeric” ILT-like polypeptides corresponded with higher molecular mass species. The material in the peaks is further analysed by SDS-PAGE.

Samples from the three peaks are pre-treated with standard SDS sample buffer (BioRad) without DTT (non-reducing) or with 15 mM DTT (reducing), and are run on a gradient 4-20% PAGE and stained with Coomassie blue stain. Under non-reducing conditions, the material in the three peaks appears as the cross-linked (ILT-PEG-ILT) species, an intermediate species (ILT-PEG) and the non-modified ILT-2 respectively.

The ability of these soluble high affinity ILT-like polypeptide dimers to bind Class I pMHC is confirmed using the Biacore-based method detailed in Example 4

### Example 10

### Tetramerisation of ILT-2 Polypeptides

Soluble high affinity ILT-like polypeptides containing an additional cysteine residue at the C-terminus are tetramerised using a tetrameric maleimide-PEG (4arm MAL-PEG, MW 20 KD, Shearwater Corporation). The maleimide groups on the termini of this linker confer free thiol binding specificity to the linker. Prior to cross-linking the soluble high affinity ILT-like polypeptides are pre-treated with a reducing agent, 0.1 mM DTT (room temperature, overnight), in order to liberate the free cysteine on the soluble ILT-2 polypeptides. This low concentration of reducing agent is used to selectively reduce the exposed C-terminal cysteine residue. The soluble high affinity c50 ILT-like polypeptides are then re-purified by gel-filtration chromatography (Superdex 75) in PBS buffer. The soluble high affinity ILT-like polypeptides are then re-concentrated using a 10 kDa cut-off centrifugal membrane concentrator (VivaScience, Satorius). Tetramerisaton is achieved by the stepwise addition of the 4arm MAL-PEG (10 mM in DMF) at an approximately 4:1 (protein to cross-linker) molar ratio and subsequent incubation for 2 hours at room temperature. The product is then purified using Superdex 75 HR10/30 gel-filtration column pre-equilibrated in PBS. The eluted fractions are further analysed by SDS-PAGE.

Samples from the fractions are pre-treated with standard SDS sample buffer (BioRad) without DTT (non-reducing) or with 15 mM DTT (reducing), and are run on a gradient 4-20% PAGE and stained with Coomassie blue stain.

The ability of these tetramers to bind Class I pMHC is confirmed using the Biacore-based method detailed in Example 4

### Example 11

### ELISPOT Assay for Assessing In-Vitro Inhibition of Cyto-Toxic T Cell (CTL) Activation by High Affinity c83 ILT-Like Polypeptides

The following method provides a means of assessing the ability of soluble high affinity c83 (SEQ ID NO: 19) ILT-like polypeptides to inhibit CD8 co-receptor mediated T cell activation.

### Reagents:

Assay media: 10% FCS (heat-inactivated, Gibco, cat#10108-165), 88% RPMI 1640 (Gibco, cat#42401-018), 1% glutamine (Gibco, cat#25030-024) and 1% penicillin/streptomycin (Gibco, cat#15070-063).

Wash buffer: 0.01 M PBS/0.05% Tween 20 (1 sachet of Phosphate buffered saline with Tween 20, pH7.4 from Sigma, Cat. #P-3563 dissolved in 1 litre distilled water gives final composition 0.01 M PBS, 0.138 M NaCl, 0.0027 M KCl, 0.05% Tween 20).

PBS (Gibco, cat#10010-015).

Diaclone EliSpot kit (IDS) EliSpot kit contains all other reagents required i.e. capture and detection antibodies, skimmed milk powder, BSA, streptavidin-alkaline phosphatase, BCIP/NBT solution (Human IFN-γ PVDF Eli-spot 20×96 wells with plates (IDS cat#DC-856.051.020, DC-856.000.000.

The following method is based on the manufacturers instructions supplied with each kit but contains some alterations.

### Method

100 μl capture antibody was diluted in 10 ml sterile PBS per plate. 100 μl diluted capture antibody was aliquoted into each well and left overnight at 4° C., or for 2 hr at room temperature. The plates were then washed three times with 450 μl wash buffer, Ultrawash 96-well plate washer, (Thermo Life Sciences) to remove excess capture antibody. 100 μl of 2% skimmed milk was then added to each well. (One vial of skimmed milk powder as supplied with the EliSpot kit was dissolved in 50 ml sterile PBS). The plates were then incubated at room temperature for two hours before washing washed a further three times with 450 μl wash buffer, Ultrawash 96-well plate washer, (Thermo Life Sciences)

Mel 624 target cells were detached from their tissue culture flasks using trypsin, washed once by centrifugation (280×g for 10 minutes) in assay media and resuspended at 1×10⁶/ml in the same media. 50 ul of this suspension was then added to the assay plate to give a total target cell number of 50,000 cells/well.

A MART-1 specific T cell clone (KA/C5) (effector cell line) was harvested by centrifugation (280×g for 10 min) and resuspended at 1×10⁴/ml in assay media to give 500 cells/well when 50 μl was added to the assay plate.

Soluble high affinity c83 (SEQ ID NO: 19) ILT-like polypeptide was diluted in assay media at a 3× concentration to give a 1× final when 50 ul was added to the plate in a final volume of 150 μl. The concentration range of ILT-2 monomer tested was 0.84 μM-0.25 nM.

Wells containing the following were then prepared, (the final reaction volume in each well was 100 μl):

### Test Samples (Added in Order)

50 μl Mel 624 target cells
50 ul of the desired concentration of c83 ILT-like polypeptide.
50 ul T cell clone effector cells.

### Negative Controls

50 μl target cells
50 μl of the highest concentration of c83 ILT-like polypeptide
50 μl assay media

### OR

50 μl effector cells
50 μl of the highest concentration of c83 ILT-like polypeptide
50 μl assay media

### Positive Controls

50 μl Mel 624 target cells
50 μl effector cells
50 μl assay media

### OR

### To Show CD8 Dependency

50 μl Mel 624 target cells
50 μl effector cells
50 μl containing 100 μg/ml HB230 anti CD8 antibody

The plates were then incubated overnight at 37° C./5% CO₂. The plates were then washed six times with wash buffer before tapping out excess buffer. 550 μl distilled water was then added to each vial of detection antibody supplied with the ELISPOT kit to prepare a diluted solution. 100 μl of the diluted detection antibody solution was then further diluted in 10 ml PBS/1% BSA per plate and 100 μl of the diluted detection antibody solution was aliquoted into each well. The plates were then incubated at room temperature for 90 minutes.

After this time the plates were washed three times with wash buffer (three times with 450 μl wash buffer, Ultrawash 96-well plate washer (Thermo Life Sciences) and tapped dry. 10 μl streptavidin-Alkaline phosphatase was then diluted with 10 ml with PBS/1% BSA per plate and 100 μl of the diluted streptavidin was added to each well and incubated at room temperature for 1 hr. The plates were then washed again three times with 450 μl wash buffer and tapped dry.

100 μl of the BCIP/NBT supplied solution was added to each well and the plates are covered in foil and left to develop for 5-15 min. The plates were checked regularly during this period for spot formation in order to decide when to terminate the reaction.

The plates were then washed thoroughly in tap water and shaken before being taken apart and left to dry on the bench.

Once dry the plates were read using an ELISPOT reader (Autoimmune Diagnotistika, Germany).

The number of spots that appeared in each well is proportional to the number of T cells activated. Therefore, any decrease in the number of spots in the wells containing the soluble high affinity c83 (SEQ ID NO: 19) ILT-like polypeptide indicates inhibition of CD8 co-receptor-mediated CTL activation.

### Results

As shown in FIG. 7 the high affinity c83 (SEQ ID NO: 19) ILT-2 polypeptide is effective at inhibiting CTL activation The IC₅₀ values calculated from the data from the three determinations shown in FIG. 7 for the inhibition of CTLs by the high affinity c83 ILT-2 polypeptide were 76 nM, 94 nM and 117 nM. (Average IC₅₀ value=96 nM)

1. A polypeptide having the property of binding to a given Class I with a K_{D} of less than or equal to 1 μM and/or an off-rate (k_{off}) of 2 S⁻¹ or slower AND said polypeptide has at least a 90% identity and/or 95% similarity to SEQ ID NO: 19 AND said polypeptide inhibits CD8 binding to the given pMHC to a greater extent than the polypeptide SEQ ID NO: 3, CHARACTERISED IN THAT said polypeptide comprises an amino acid sequence selected from one of SEQ ID NOs 6 to 69.

2. A variation of a polypeptide as claimed in claim 1 which has 196L and 198L instead of 196D and/or 198D, using the numbering of SEQ ID NO: 3.

3. The polypeptide of claim 1, wherein the polypeptide is a mutated human ILT-2 having one or more of amino acids corresponding to amino acids selected from the group consisting of 23V, 35E, 126Q, 127V, 128A, 129F, 130D, 170R, 179D, 180S, 181N, 182S, 187S and 189P of SEQ ID NO: 3 mutated.

4. The polypeptide of claim 3, wherein the polypeptide comprises one or more mutations selected from the group consisting of 23V→L, 35E→Q, 126Q→P, 126Q→M, 127V→W, 127V→F, 128A→D, 128A→S, 128A→T, 128A→Y, 128A→V, 128A→L, 128A→Q, 128A→I, 129F→A, 129F→T, 129F→S, 129F→V, 130D→E, 170R→K, 179D→P, 179D→N, 179D→M, 179D→T, 179D→G, 180S→I, 180S→A, 180S→N, 180S→D, 180S→W, 180S→R, 180S→E, 181N→W, 181N→F, 181N→Y, 182S→T, 182S→A, 182S→W, 182S→F, 182S→L, 187S→T, 189P→G, 189P→M and 189P→S using the numbering of SEQ ID NO: 3.

5. The polypeptide of claim 1, wherein the polypeptide is a mutated human ILT 2 comprising one or more of mutations selected from the group consisting of 188L→R, 188L→S 188L→T and 188L→Q using the numbering of SEQ ID NO: 3

6. The polypeptide as claimed in claim 3 comprising mutations selected from the group consisting of 19Q→M, 20G→D, 21→Q, 83A→S, 84G→Q, 85R→W, 87E→A, 99A→V, 179D→M, 181N→W, 182S→A, 196L→D and 198L→D using the numbering of SEQ ID NO: 3.

7. (canceled)

8. The polypeptide as claimed in claim 1 comprising a C-terminal reactive site for covalent attachment of a desired moiety.

9. The polypeptide as claimed in claim 8, wherein said reactive site is a cysteine residue.

10. The polypeptide as claimed in claim 1 wherein the polypeptide is associated with at least one polyalkylene glycol chain(s).

11. The polypeptide as claimed in claim 10 wherein the polyalkylene glycol chain(s) is/are covalently linked to the polypeptide.

12. (canceled)

13. The A polypeptide as claimed in claim 1 wherein the polypeptide is covalently linked to a therapeutic agent or detectable label.

14. The polypeptide as claimed in claim 13 wherein the therapeutic agent is covalently linked to the C terminus of the polypeptide.

15. The polypeptide as claimed in claim 13 wherein the therapeutic agent is an immune effector molecule.

16. The polypeptide as claimed in claim 15 wherein the immune effector molecule is selected from the group consisting of a cytokine, IL-4, IL-10 and IL-13.

17. (canceled)

18. A multivalent complex comprising at least two polypeptides as claimed in claim 1, said multivalent complex having a K_{D} for a given Class I pMHC of less than or equal to 1 μM and/or an off-rate (k_{off}) for the said given Class I pMHC of 2 S⁻¹ or slower.

19. The multivalent complex as claimed in claim 18 wherein the polypeptides are linked by a non-peptidic polymer chain or a peptidic linker sequence.

20. The multivalent complex as claimed in claim 19 wherein the polymer chain or peptidic linker sequence extends between amino acid residues of each polypeptide which are not located in the Class I pMHC binding domain of the polypeptides.

21. The multivalent complex as claimed claim 19 in which the polypeptides are linked by a polyalkylene glycol chain or a peptidic linker derived from a human multimerisation domain.

22. The multivalent complex as claimed in claim 21 wherein a divalent alkylene spacer radical is located between the polyalkylene glycol chain and its point of attachment to a polypeptide of the complex.

23. (canceled)

24. (canceled)

25. The multivalent complex as claimed in claim 18 wherein (i) at least one of said polypeptide is associated with a therapeutic agent.

26. (canceled)

27. An isolated nucleic acid encoding the polypeptide as claimed claim 1.

28. (canceled)

29. A vector incorporating the nucleic acid as claimed in claim 27.

30. (canceled)

31. A pharmaceutical composition comprising the polypeptide as claimed in claim 1, together with a pharmaceutically acceptable carrier.

32. (canceled)

33. (canceled)

34. A pharmaceutical composition comprising the polypeptide as claimed in claim 13, together with a pharmaceutically acceptable carrier.

35. (canceled)

36. (canceled)

37. A method of treating an autoimmune disease comprising administering to a subject suffering from such autoimmune disease an effective amount of the polypeptide as claimed in claim 1.

38. (canceled)

39. A method of treating an autoimmune disease comprising administering to a subject suffering from such autoimmune disease an effective amount of the polypeptide as claimed in claims 13.

40-44. (canceled)


SEQUENCE LISTING (72 sequences)

SEQ ID NO: 1 - protein, 650 aa, Homo sapiens
MTPILTVLICLGLSLGPRTHVQAGHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRRKATEPPPSQEGPSPAVPSIYATLAIH

SEQ ID NO: 2 - DNA, 2984 bp, Homo sapiens
gaggaggaacagaaaagaaaagaaaagaaaaagtgggaaacaaataatctaagaatgaggagaaagcaagaagagtgacccccttgtgggcactccattggttttatggcgcctctactttctggagtttgtgtaaaacaaaaatattatggtctttgtgcacatttacatcaagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggcccccggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgacccttagacggaaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaaaaaaaaaaaaaaa

SEQ ID NO: 3 - protein, 198 aa, artificial sequence
  Soluble two domain form of WT human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 4 - DNA, 690 bp, artificial sequence
  DNA encoding a soluble two domain form of WT human ILT-2
atggggcacctccccaagcccaccctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctagcggccgcaggtggcggtactagtactgttgaaagttgtttagcaaaaccccatacagaaaattcatttactaacgtctggaaagacgacaaaact

SEQ ID NO: 5 - DNA, 615 bp, artificial sequence
  DNA encoding a soluble two domain form of WT human ILT-2 as inserted into the pGMT7 vector including restriction enzyme recognition sites
tatacatatgggtcatcttccaaaaccaactctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagccgcaggtggtggtacaggtgctatgcttatgactcgaactctccctatgagtggtctctacccagtgatctcctggagctcctggtcctataagcttgaattcc

SEQ ID NO: 6 - protein, 198 aa, artificial sequence
  Soluble two domain form of high affinity ILT-like polypeptide
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDG

SEQ ID NO: 7 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSLPSDLLELDVDG

SEQ ID NO: 8 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVAWAPYEWSLPSDLLELDVDG

SEQ ID NO: 9 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMNWAPYEWSLPSDLLELDVDG

SEQ ID NO: 10 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYTDYSPYEWSLPSDLLELDVDG

SEQ ID NO: 11 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVDWFPYEWSLPSDLLELDVDG

SEQ ID NO: 12 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVSYWPYEWSLPSDLLELDVDG

SEQ ID NO: 13 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPAWTPYEWSLPSDLLELDVDG

SEQ ID NO: 14 - protein, 200 aa, artificial sequence
  Soluble two domain form of high affinity ILT-like polypeptide
MAQHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVDFDGFILCKEGEDEHPQCLNSQPHARGSSRAIFSVGPVSPSRRWWYRCYAYDSNSPYEWSLPSDLLELDVDG

SEQ ID NO: 15 - protein, 200 aa, artificial sequence
  Soluble two domain form of high affinity ILT-like polypeptide
MAQHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVSFDGFILCKEGEDEHPQCLNSQPHARGSSRAIFSVGPVSPSRRWWYRCYAYDSNSPYEWSLPSDLLELDVDG

SEQ ID NO: 16 - protein, 200 aa, artificial sequence
  Soluble two domain form of high affinity ILT-like polypeptide
MAQHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVTFDGFILCKEGEDEHPQCLNSQPHARGSSRAIFSVGPVSPSRRWWYRCYAYDSNSPYEWSLPSDLLELDVDG

SEQ ID NO: 17 - protein, 200 aa, artificial sequence
  Soluble two domain form of high affinity ILT-like polypeptide
MAQHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAYDGFILCKEGEDEHPQCLNSQPHARGSSRAIFSVGPVSPSRRWWYRCYAYDSNSPYEWSLPSDLLELDVDG

SEQ ID NO: 18 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWAPYEWSLPSDLLELDVDG

SEQ ID NO: 19 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDG

SEQ ID NO: 20 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPDFTPYEWSLPSDLLELDVDG

SEQ ID NO: 21 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPWFTPYEWSLPSDLLELDVDG

SEQ ID NO: 22 - protein, 199 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYGRWLPYEWSLPSDLLELDVD

SEQ ID NO: 23 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVEWLPYEWSLPSDLLELDVDG

SEQ ID NO: 24 - protein, 198 aa, artificial sequence
  Soluble two domain form of high affinity ILT-like polypeptide
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYPIWTPYEWSLPSDLLELDVDG

SEQ ID NO: 25 - protein, 198 aa, artificial sequence
  Soluble two domain form of high affinity ILT-like polypeptide
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYVAWAPYEWSLPSDLLELDVDG

SEQ ID NO: 26 - protein, 198 aa, artificial sequence
  Soluble two domain form of high affinity ILT-like polypeptide
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMNWAPYEWSLPSDLLELDVDG

SEQ ID NO: 27 - protein, 198 aa, artificial sequence
  Soluble two domain form of high affinity ILT-like polypeptide
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYPAWTPYEWSLPSDLLELDVDG

SEQ ID NO: 28 - protein, 198 aa, artificial sequence
  Soluble two domain form of high affinity ILT-like polypeptide
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMSWAPYEWSLPSDLLELDVDG

SEQ ID NO: 29 - protein, 198 aa, artificial sequence
  Soluble two domain form of high affinity ILT-like polypeptide
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMSWSPYEWSLPSDLLELDVDG

SEQ ID NO: 30 - protein, 200 aa, artificial sequence
  Soluble two domain form of high affinity ILT-like polypeptide
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSLPSDLLELDVDG

SEQ ID NO: 31 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSLPSDLLELDVDG

SEQ ID NO: 32 - protein, 200 aa, artificial sequence
  Soluble two domain form of high affinity ILT-like polypeptide
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVAWAPYEWSLPSDLLELDVDG

SEQ ID NO: 33 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYVAWAPYEWSLPSDLLELDVDG

SEQ ID NO: 34 - protein, 200 aa, artificial sequence
  Soluble two domain form of high affinity ILT-like polypeptide
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMNWAPYEWSLPSDLLELDVDG

SEQ ID NO: 35 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYMNWAPYEWSLPSDLLELDVDG

SEQ ID NO: 36 - protein, 200 aa, artificial sequence
  Soluble two domain form of high affinity ILT-like polypeptide
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPAWTPYEWSLPSDLLELDVDG

SEQ ID NO: 37 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYPAWTPYEWSLPSDLLELDVDG

SEQ ID NO: 38 - protein, 200 aa, artificial sequence
  Soluble two domain form of high affinity ILT-like polypeptide
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWAPYEWSLPSDLLELDVDG

SEQ ID NO: 39 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWAPYEWSLPSDLLELDVDG

SEQ ID NO: 40 - protein, 200 aa, artificial sequence
  Soluble two domain form of high affinity ILT-like polypeptide
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDG

SEQ ID NO: 41 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDG

SEQ ID NO: 42 - protein, 198 aa, artificial sequence
  Soluble two domain form of high affinity ILT-like polypeptide
MHLPKPTLWAEPGSVITLQSPLTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDG

SEQ ID NO: 43 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSYSPYEWSLPSDLLELDVDG

SEQ ID NO: 44 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPAYTPYEWSLPSDLLELDVDG

SEQ ID NO: 45 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSNSPYEWSLPSDLLELDVDG

SEQ ID NO: 46 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSWSPYEWSLPSDLLELDVDG

SEQ ID NO: 47 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSNAPYEWSLPSDLLELDVDG

SEQ ID NO: 48 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSWAPYEWSLPSDLLELDVDG

SEQ ID NO: 49 - protein, 198 aa, artificial sequence
  Soluble two domain form of high affinity ILT-like polypeptide
MHLPKPTLWAEPGSVITLQSPLTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMSWSPYEWSLPSDLLELDVDG

SEQ ID NO: 50 - protein, 198 aa, artificial sequence
  Soluble two domain form of high affinity ILT-like polypeptide
MHLPKPTLWAEPGSVITLQSPLTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMSWAPYEWSLPSDLLELDVDG

SEQ ID NO: 51 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSLGSDLLELDVDG

SEQ ID NO: 52 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSQMSDLLELDVDG

SEQ ID NO: 53 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWTRGSDLLELDVDG

SEQ ID NO: 54 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSTSSDLLELDVDG

SEQ ID NO: 55 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSSGSDLLELDVDG

SEQ ID NO: 56 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWTLGSDLLELDVDG

SEQ ID NO: 57 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWTRGSDLLELDVDG

SEQ ID NO: 58 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSQMSDLLELDVDG

SEQ ID NO: 59 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSSGSDLLELDVDG

SEQ ID NO: 60 - protein, 199 aa, artificial sequence
  Soluble two domain form of high affinity ILT-like polypeptide
MGHLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWTRGSDLLELDVDG

SEQ ID NO: 61 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPWVADGFILCKEGEDEHPQCLNSQPHARGSSRAIFSVGPVSPSRRWWYRCYAYMSWSPYEWSLPSDLLELDVDG

SEQ ID NO: 62 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFLTDGFILCKEGEDEHPQCLNSQPHARGSSRAIFSVGPVSPSRRWWYRCYAYMSWSPYEWSLPSDLLELDVDG

SEQ ID NO: 63 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPWLADGFILCKEGEDEHPQCLNSQPHARGSSRAIFSVGPVSPSRRWWYRCYAYMSWSPYEWSLPSDLLELDVDG

SEQ ID NO: 64 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDG

SEQ ID NO: 65 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFIAEGFILCKEGEDEHPQCLNSQPHARGSSRAIFSVGPVSPSRRWWYRCYAYMSWSPYEWSLPSDLLELDVDG

SEQ ID NO: 66 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFLSDGFILCKEGEDEHPQCLNSQPHARGSSRAIFSVGPVSPSRKWWYRCYAYMSWSPYEWSLPSDLLELDVDG

SEQ ID NO: 67 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFVSDGFILCKEGEDEHPQCLNSQPHARGSSRAIFSVGPVSPSRRWWYRCYAYMSWSPYEWSLPSDLLELDVDG

SEQ ID NO: 68 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFIADGFILCKEGEDEHPQCLNSQPHARGSSRAIFSVGPVSPSRRWWYRCYAYMSWSPYEWSLPSDLLELDVDG

SEQ ID NO: 69 - protein, 199 aa, artificial sequence
  Soluble two domain form of high affinity ILT-like polypeptide
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSMFLVDGFILCKEGEDEHPQCLNSQPHARGSSRAIFSVGPVSPSRRWWYRCYAYMSWSPYEWSLPSDLLELDVDG

SEQ ID NO: 70 - DNA, 3111 bp, artificial sequence
  DNA sequence of a pGMT7-derived vector into which DNA encoding the amino acid sequences of soluble two domain forms of high affinity ILT-like polypeptides can be inserted
gatctcgatcccgcgaaattaatacgactcactatagggagaccacaacggtttccctctagaaataattttgtttaactttaagaaggagatatacatatgggatccatggtaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 71 - protein, 9 aa, Homo sapiens
YLSGANLNL

SEQ ID NO: 72 - protein, 200 aa, artificial sequence
  Soluble two domain form of high affinity ILT-like polypeptide
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVNFWPYEWSLPSDLLELDVDG